SEQUENCE LISTING (21 sequences)

SEQ ID NO: 1 - protein, 20 aa, synthetic construct
QIVTTNVRLKQQWVDYNLKW

SEQ ID NO: 2 - protein, 20 aa, synthetic construct
AIVKFTKVLLQYTGHITWTP

SEQ ID NO: 3 - protein, 34 aa, synthetic construct
STHVMPNWVRKVFIDTIPNIMFFSTMKRPSREKQ

SEQ ID NO: 4 - protein, 19 aa, synthetic construct
IIGTLAVFAGRLIELNQQG

SEQ ID NO: 5 - protein, 15 aa, synthetic construct
IDGQFGVAYDANVLV

SEQ ID NO: 6 - protein, 15 aa, synthetic construct
ENGEWAIDFCPGVIR

SEQ ID NO: 7 - protein, 15 aa, synthetic construct
IRRKPLFYVINIIVP

SEQ ID NO: 8 - protein, 22 aa, synthetic construct
GAAAPEVRCCVDAVNFVAESTR

SEQ ID NO: 9 - protein, 39 aa, synthetic construct
KSYCEIIVTHFPFDEQNCSMKLGTWTYDGSVVAINPESD

SEQ ID NO: 10 - protein, 26 aa, synthetic construct
MKSDQESNNAAAEWKYVAMVMDHILL

SEQ ID NO: 11 - protein, 14 aa, synthetic construct
FLLVIVELIPSTSS

SEQ ID NO: 12 - protein, 17 aa, synthetic construct
AIFKSYCEIIVTHFPFD

SEQ ID NO: 13 - protein, 16 aa, synthetic construct
EDHRQVVEVTVGLQLI

SEQ ID NO: 14 - protein, 16 aa, synthetic construct
IHIPSEKIWRPDLVLY

SEQ ID NO: 15 - protein, 16 aa, synthetic construct
WNPDDYGGVKKIHIPS

SEQ ID NO: 16 - protein, 16 aa, synthetic construct
LKQQWVDYNLKWNPDD

SEQ ID NO: 17 - protein, 18 aa, synthetic construct
YNLKWNPDDYGGVKKIHI

SEQ ID NO: 18 - protein, 18 aa, synthetic construct
IEGIKYIAETMKSDQESN

SEQ ID NO: 19 - protein, 46 aa, synthetic construct
QIVTTNVRLKQQWVDYNLKWNPDDYGGVKKIHIPSEKIWRPDLVLY

SEQ ID NO: 20 - protein, 38 aa, synthetic construct
AIVKFTKVLLQYTGHITWTPPAIFKSYCEIIVTHFPFD

SEQ ID NO: 21 - protein, 50 aa, synthetic construct
ENGEWAIDFCPGVIRRHHGGATDGPGETDVIYSLIIRRKPLFYVINIIVP

(19) United States
(12) Patent Application Publication
(10) Pub. No.: US 2026/0284161 A1
(43) Pub. Date: Sep. 24, 2026

(54) TOLERIZING IMMUNE MODIFYING NANOPARTICLES FOR MYASTHENIA GRAVIS TREATMENT

(71) Applicant: COUR PHARMACEUTICALS DEVELOPMENT COMPANY INC., Skokie, IL (US)

(72) Inventors: Adam Elhofy, Chicago, IL (US); Greta Wodarcyk, Northbrook, IL (US); Michael Boyne, Skokie, IL (US)

(21) Appl. No.: 19/168,118

(22) PCT Filed: Mar. 25, 2024
(86) PCT No.: PCT/US2024/021269

§ 371 Date: Sep. 23, 2025

Related U.S. Application Data
(60) Provisional application No. 63/492,165, filed on Mar. 24, 2023.

(51) Int. Cl. A61K 39/00; A61K 9/51; A61K 39/39; A61P 21/04; A61P 37/06; C07K 14/705

(52) U.S. Cl. CPC A61K 39/0008; A61K 9/5153; A61K 39/39; A61P 21/04; A61P 37/06; C07K 14/705; A61K 2039/545; A61K 2039/55555; A61K 2039/577; A61K 2039/70

(57) ABSTRACT

The present application is directed, in general, to compositions comprising tolerizing immune modifying particles encapsulating Myasthenia Gravis (MG) associated antigens, methods of treating MG using tolerizing immune modifying nanoparticles encapsulating MG associated antigens, and a process for the preparation of tolerizing immune modifying nanoparticles encapsulating MG antigens.

### CROSS REFERENCE TO RELATED APPLICATIONS

The present application claims the priority benefit of U.S. Provisional Patent Application No. 63/492,165, filed Mar. 24, 2024, hereby incorporated by reference in its entirety.

### FIELD OF THE DISCLOSURE

The present application is directed, in general, to tolerizing immune modifying particles comprising antigens associated with myasthenia gravis (TIMP-MG), methods of treating myasthenia gravis using TIMP-MG, and methods of manufacturing TIMP-MG.

### BACKGROUND

Myasthenia Gravis (MG) is a rare, autoimmune disorder of the neuromuscular junction characterized by fluctuating muscle weakness and abnormal fatigability¹.

MG is a T cell driven autoimmune disease primarily directed towards acetylcholine (AChR) receptor. Activated T cells drive T cell mediated B cell antibody (Ab) production specific for AChR² that prevents AChR from binding acetylcholine. The combination of T cell and B cell activation results in IgG Ab and complement deposits at the neuromuscular junction, leading to impaired neuromuscular transmission. Rare forms of MG are due to autoantibodies targeting other components such a muscle-specific kinase (MuSK), lipoprotein receptor-related protein (LRP4), Agrin, Cortactin, Titin and/or Ryanodine³.

Disease progression can lead to life-threatening complications including myasthenic crisis, tumors of the thymus gland, thyroid disorders and/or other autoimmune conditions.

The global prevalence of MG ranges from 40 to 180 per million⁴ with an estimated annual incidence of 1.74 to 12 cases per million persons 4.

There is currently no cure for MG and the standard of care is focused on alleviating symptoms with the use of steroids or immunosuppressants that only have a temporary effect and are associated with significant side-effects such as increased risks of infections and even death.

### SUMMARY OF THE DISCLOSURE

Tolerizing immune modifying particles (TIMPs), comprising one or more antigens, have been previously described for the treatment of immune-mediated disorders (e.g autoimmune diseases and allergies) via induction of antigen-specific immune tolerance (WO20131319253 and WO2015023796 incorporated herein by reference). Encapsulation of one or more MG associated antigens within TIMP core is an advantage as it ensures delivery of encapsulated proteins to APCs safely and effectively without inducing immune activation. In several preclinical models of autoimmune diseases and allergies, TIMPs have demonstrated efficacy at inducing T-cell tolerance. Induction of antigen-specific tolerance to MG autoantigens using TIMPs encapsulating MG-associated antigens (TIMP-MG) could potentially cure MG.

The present disclosure describes TIMP-MG compositions, methods of treating MG in a subject comprising administering to the subject TIMP-MG, and methods of manufacturing TIMP-MG for the treatment of MG.

Provided herein is a TIMP-MG composition comprising a negatively charged TIMP-MG particle encapsulating an antigen, wherein the antigen comprises one or more MG associated antigens and/or a portion thereof, or combinations of antigens or portions thereof. In various embodiments, the TIMP-MG particle comprises a polymer and has a negative zeta potential. In various embodiments, the polymer is a biodegradable polymer.

In various embodiments, the TIMP-MG particles comprise a polymer selected from the group consisting of polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid, polystyrene, diamond, a liposome, PEG, cyclodextran, a lipid or a metal such as Iron (Fe), zinc (Zn), cadmium (Cd), Gold, or Silver, or combinations thereof.

In various embodiments, the polymer is a co-polymer. In various embodiments, the co-polymer has varying molar ratios of constituent polymers. In various embodiments, the molar ratio is 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0, including all values and ranges that lie between these values.

In various embodiments, the TIMP-MG particles comprise poly(lactic-co-glycolic acid) (PLGA). In various embodiments, the particle comprises about 50:50, about 80:20 to about 100:0 polylactic acid:polyglycolic acid or from about 50:50, about 80:20 to about 100:0 polyglycolic acid:polylactic acid. In various embodiments, the particle comprises 50:50 polylactic acid:polyglycolic acid. In various embodiments, the particle comprises polylactic acid:polyglycolic acid from about 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0, including all values and ranges that lie in between these values.

In various embodiments, the TIMP-MG particles have a negative zeta potential. In various embodiments, the zeta potential of the particles is from about −100 mV to about 0 mV, from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and ranges therein. In various embodiments, the TIMP-MG particles have a negative zeta potential of between −30 mV to −80 mV. In various embodiments, the TIMP-MG particles have a negative zeta potential of between −30 mV to −60 mV. In various embodiments, the negative zeta potential is achieved by surface functionalization of the TIMP-MG particle. In various embodiments, the surface functionalization is carboxylation.

In various embodiments, the size, or diameter, of TIMP-MG particles is between 0.05 μm to about 10 μm. In various embodiments, the diameter of TIMP-MG particles is between 0.1 μm and about 10 μm. In various embodiments, the diameter of TIMP-MG particles is between 0.1 μm and about 5 μm. In various embodiments, the diameter of TIMP-MG particles is between 0.1 μm and about 3 μm. In various embodiments, the diameter of TIMP-MG particles is between 0.3 μm and about 5 μm. In various embodiments, the diameter of TIMP-MG particles is about 0.3 μm to about 3 μm. In various embodiments, the diameter of TIMP-MG particles is between about 0.3 μm to about 1 μm. In various embodiments, the diameter of TIMP-MG particles is between about 0.4 μm to about 1 μm. In various embodiments, the TIMP-MG particles have a diameter of about 100 to 10000 nm, about 100 to 5000 nm, about 100 to 3000 nm, about 100 to 2000 nm, about 100 to 1500 nm, about 300 to 5000 nm, about 300 to 3000 nm, about 300 to 1000 nm, about 300 to 800 nm, about 400 to 800 nm, or about 200 to 700 nm. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and ranges therein. In various embodiments, the diameter of the negatively charged particle is between 400 nm to 800 nm. In various embodiments, the diameter of the negatively charged particle is between 350 nm to 800 nm.

In various embodiments, the TIMP-MG particles have a homogenous size distribution. In various embodiments, the TIMP-MG particles have a homogenous size distribution wherein at least 90% of the particles have a diameter of between 0.05 μm and about 10 μm, between 0.1 μm and about 10 μm, 0.1 μm and about 5 μm, 0.1 μm and about 3 μm, 0.3 μm and about 5 μm, 0.3 μm to about 3 μm including all values and ranges therein. In various embodiments, the TIMP-MG particles have a homogenous size distribution wherein at least 90% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm including all values and ranges therein. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm including all values and ranges therein. In various embodiments, the TIMP-MG particles have a homogenous size distribution wherein at least 50% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm including all values and ranges therein. In various embodiments, the particles have a homogenous size distribution wherein at least 50% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm including all values and ranges therein. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm including all values and ranges therein. In various embodiments, the TIMP-MG particles have a homogenous size distribution wherein at least 10% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm including all values and ranges therein. In various embodiments, the TIMP-MG particles have a homogenous size distribution wherein at least 10% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm including all values and ranges therein. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm including all values and ranges therein.

In various embodiments, the TIMP-MG particles encapsulating one or more MG associated antigens, have a negative zeta potential of between −100 mV and 0 mV, and wherein the particle is between 100 and 1000 nm in diameter. In various embodiments, the TIMP-MG particles encapsulating one or more MG associated antigens, portions thereof or combinations thereof, wherein the size of the particles is between 400 and 800 nm and the particles have a negative zeta potential between −30 mV and −80 mV.

In various embodiments, the TIMP-MG particles encapsulate one or more MG associated antigens, portions, or combinations thereof. In various embodiments, the antigen comprises one or more proteins, peptides, antigenic epitopes, or combinations thereof. In various embodiments, the TIMP-MG particles encapsulate one or more polynucleotides encoding MG associated antigens. In some embodiments, the polynucleotides comprise DNA, RNA, messenger RNA (mRNA), or circular RNA.

In various embodiments, the MG associated antigen is acetylcholine receptor (AChR) protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3), portions thereof, and/or one or more antigenic epitopes thereof. In various embodiments, the one or more MG associated antigens are selected from the listing in Table 1. In various embodiments, the antigen is the AChR protein subunit and/or a portion thereof. In various embodiments, the antigen is the AChR antigen and/or a portion thereof, or one or more antigenic epitopes thereof selected from the group consisting of SEQ ID NOS: 1 to 21 (Table 2). In various embodiments, the one or more antigens encapsulated within the particles comprises a combination of SEQ ID NO: 3, SEQ ID NO: 5, SEQ ID NO: 8, SEQ ID NO: 13, SEQ ID NO: 19, SEQ ID NO: 20, and/or SEQ ID NO: 21 listed in Table 2.

In various embodiments, the TIMP-MG particles encapsulate one or more MG associated antigens, have a negative zeta potential of between −100 mV and 0 mV, and wherein the particles are between 100 and 1000 nm in diameter. In various embodiments, the TIMP-MG particles encapsulate antigens selected from the group consisting listed in Table 1, wherein the size of the particle is between 400 and 800 nm, and the particles have a negative zeta potential between-30 mV and −80 mV. In various embodiments, the TIMP-MG particles encapsulate antigens selected from the group consisting of SEQ ID NOS: 1 to 21 (Table 2), portions or combinations thereof, wherein the size of the particle is between 400 and 800 nm, and the particles have a negative zeta potential between −30 mV and −80 mV. In various embodiments, the TIMP-MG particles encapsulate one or more MG associated antigens comprising SEQ ID NO: 3, SEQ ID NO: 5, SEQ ID NO: 8, SEQ ID NO: 13, SEQ ID NO: 19, SEQ ID NO: 20, and/or SEQ ID NO: 21 (Table 2), portions thereof or combinations thereof, and wherein the size of the particle is between 400 and 800 nm and the TIMP-MG particles have a negative zeta potential between −30 mV and −80 mV.

Provided herein is a method of treating MG in a subject comprising administering to the subject TIMP-MG, wherein TIMP-MG is administered at a dose of 0.001 mg/kg to 12 mg/kg. In various embodiments, TIMP-MG is administered at a dose determined based on the subject's weight. In various embodiments, TIMP-MG is administered at a fixed dose of between 0.1 mg to 800 mg. In various embodiments, the TIMP-MG is administered at a dose from about from about 0.001 to about 10 mg/kg, from about 0.005 to about 12 mg/kg, from about 0.01 to about 12 mg/kg, from about 0.05 to about 12 mg/kg, from about 0.1 to about 12 mg/kg, from about 0.5 to about 10 mg/kg, from about 1 mg/kg to about 8 mg/kg, from about 1.5 to about 10 mg/kg, from about 2 mg/kg to about 12 mg/kg, from about 2 mg/kg to about 10 mg/kg, from about 3 mg/kg to about 10 mg/kg, from about 4 to about 10 mg/kg, from about 4 to about 12 mg/kg, or from about 5 to about 12 mg/kg. In various embodiments, TIMP-MG is administered at a dose of about 0.001 mg/kg, about 0.0025 mg/kg, about 0.005 mg/kg, about 0.01 mg/kg, about 0.025 mg/kg, about 0.05 mg/kg, about 0.1 mg/kg, about 0.25 mg/kg, about 0.5 mg/kg, about 1.0 mg/kg, about 2.0 mg/kg, about 3.0 mg/kg, about 4.0 mg/kg, about 5 mg/kg, about 6 mg/kg, about 8.0 mg/kg, about 10 mg/kg, or about 12 mg/kg. In various embodiments, TIMP-MG is administered at a fixed dose between 0.1 mg and 800 mg. Also provided herein is a method of reducing immune response to MG associated antigens in a subject suffering from MG comprising administering to the subject TIMP-MG, wherein TIMP-MG is administered at a fixed dose between 0.1 mg and 800 mg. In various embodiments, TIMP-MG is administered at a fixed dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In various embodiments, TIMP-MG is administered in a single dose or in multiple doses. In various embodiments, TIMP-MG is administered in two doses one-week apart. In various embodiments, TIMP-MG is administered once weekly, once every two weeks, once every three weeks, once every 4 weeks, once every two months, once every three months, once every four months, once every five months, once every 6 months, or once per year.

In various embodiments, a booster dose of TIMP-MG is administered in a single dose or in multiple doses following the loading TIMP-MG administration. In various embodiments, the booster dose is administered once weekly, once every two weeks, once every three weeks, once every 4 weeks, once every two months, once every three months, once every 4 months, once every 5 months, once every 6 months, or once per year. In various embodiments, TIMP-MG is administered in two doses one-week apart followed by a booster dose administered as a single dose once every three months.

In various embodiments, booster dose of TIMP-MG is administered at a dose of 0.001 mg/kg to 12 mg/kg. In various embodiments, the booster dose of TIMP-MG is administered at a dose from about 0.001 to 10 mg/kg, from about 0.005 to 12 mg/kg, from about 0.01 to 12 mg/kg, from about 0.05 to 12 mg/kg, from about 0.1 to 12 mg/kg, from about 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg, or about 0.001 mg/kg, 0.0025 mg/kg, 0.005 mg/kg, 0.01 mg/kg, 0.025 mg/kg, 0.05 mg/kg, 0.1 mg/kg, 0.25, 0.5 mg/kg, 1.0 mg/kg, 2.0 mg/kg, 4.0 mg/kg, 6 mg/kg, 8.0 mg/kg, 10 mg/kg, or 12 mg/kg. In various embodiments, the booster dose of TIMP-MG is administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In various embodiments, TIMP-MG is administered intravenously, subcutaneously, intramuscularly, intraperitoneally, intranasally, or orally.

In various embodiments, the disclosure provides a method of treating MG or MG related symptoms, in a subject comprising administering to the subject a composition comprising TIMP-MG alone or in combination with a therapeutic. In various embodiments, the therapeutic is a cholinesterase inhibitor, steroid, corticosteroid, nonsteroidal immunosuppressive agent, immunomodulatory agent, therapeutic plasma exchange (plasmapheresis), intravenous immunoglobulin (IVIG), chloride ion channel inhibitor, monoclonal antibody, proteasome inhibitors, cytokine and chemokine targeting therapy, microRNA inhibitor, siRNA, JAK inhibitor, BTK inhibitor, complement inhibitor, IgG degrader, chimeric antigen receptor (CAR) T-cell therapy, regulatory T-cell (Treg) therapy, hematopoietic stem cell transplant, B-cell targeting therapy, or surgical treatment. In various embodiments, the therapies are selected from the group comprising azathioprine, sirolimus, edrophonium, neostigmine, pyridostigmine, cyclosporine, mycophenolate mofetil, calcineurin inhibitor, levamisole, chlorambucil, cyclophosphamide, tacrolimus, methotrexate, eculizumab, ravulizumab, efgartigimod alfa, rozanolixizumab, zilucoplan, CD20 targeting therapy, CD19 targeting therapy, CD40/CD40L targeting therapy, B cell targeting factor (BAFF) targeting therapy, B cell maturation antigen (BCMA) targeting therapy, anti-IL6 therapy, anti-IFN therapy, anti-thymocyte globulin, factor D inhibitor, amifampridine, batoclimab, inebilizumab, nipocalimab, pozelimab, rituximab, satralizumab, tocilizumab, tofacitinib, tolebrutininb. In various embodiments the steroid or corticosteroid, is selected from the group comprising beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, oxymetazoline hydrochloride, or hydrocortisone. In various embodiments, the surgical treatment is a thymectomy. In various embodiments the therapeutic is administered prior to, concomitantly with or after the administration of TIMP-MG. In various embodiments, the therapeutic is administered 1, 2, 3, 4, 5, 6, or 7 days prior to administration of TIMP-MG. In various embodiments, the therapeutic is administered 1, 2, 3, or 4 weeks prior to administration of TIMP-MG. In various embodiments, the therapeutic is administered 1, 2, 3, 4, 5, 6, 7, 8, 9, 10, 11, or 12 prior to administration of TIMP-MG. In various embodiments, the therapeutic is administered 1, 2, 3, 4, 5, 6, 7, 8, 9, 10, 11, or 12 years prior to administration of TIMP-MG. In various embodiments, the therapeutic is administered 1, 2, 3, 4, 5, 6, or 7 days subsequent to administration of TIMP-MG. In various embodiments, the therapeutic is administered 1, 2, 3, or 4 weeks subsequent to administration of TIMP-MG. In various embodiments, the therapeutic is administered 1, 2, 3, 4, 5, 6, 7, 8, 9, 10, 11, or 12 months subsequent to administration of TIMP-MG. In various embodiments, the therapeutic is administered 1, 2, 3, 4, 5, 6, 7, 8, 9, 10, 11, or 12 years subsequent to administration of TIMP-MG.

In various embodiments, TIMP-MG is administered via intravenous infusion lasting about 0.5, 1, 2, 3, 4, 5, 6, 7, or 8 hours including all values lying within this range.

In various embodiments, TIMP-MG consists of poly(lactic co-glycolic acid) (PLGA) particles encapsulating one or more MG antigens and a suitable buffering agent or excipient. In various embodiments, TIMP-MG particles are surface functionalized. In various embodiments, TIMP-MG particles are surface functionalized by carboxylation. In various embodiments, TIMP-MG particles have a negative zeta potential. In various embodiments, the negative zeta potential of TIMP-MG particles is between about −100 mV to about 0 mV. In various embodiments, the zeta potential of the particles is from about −100 mV to about −25 mV, from about −100 to about −30 mV, from about −80 mV to about −30 mV, from about −75 mV to about-30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV.

In various embodiments, TIMP-MG is administered at a concentration of between about 0.0005 mg/mL and about 50 mg/mL. In various embodiments, TIMP-MG is administered at a concentration of about 0.0005 mg/mL, 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 3.25 mg/ml, 3.5 mg/ml, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL. In various embodiments, TIMP-MG is administered via intravenous infusion lasting about 0.5, 1, 2, 3, 4, 5, 6, 7, or 8 hours.

In various embodiments, administering TIMP-MG to a subject in need thereof, ameliorates or relieves one or more symptoms of MG. In various embodiments, the symptoms of MG are selected from the group consisting of droopy eyelids (ptosis), double vision (diplopia), difficulty making facial expressions, problems chewing, difficulty swallowing, slurred speech (dysarthria), shortness of breath, breathing difficulties, muscle fatigue, skeletal muscle weakness, neck weakness, limb weakness, dysphagia, dysphonia, weakened neuromuscular junctions, impaired synaptic signal transduction, muscle damage, anti-Musk antibodies, anti-AChR antibodies, increased levels of activated CD4+ cells when compared to a healthy subject, increased levels activated CD8+ T-cells when compared to a healthy subject, increased Myasthenia Gravis Activities of Daily Living Score, increased Quantitative MG Score, MG Quality of Life 15-revised score, and MG Composite Score.

In various embodiments, administering TIMP-MG to a subject in need thereof, ameliorates or reduces the duration and severity of an inflammatory immune response to one or more MG antigens. In various embodiments, the inflammatory immune response is a T cell response, B cell response, Th1/Th17 response, myeloid cell response, complement response, and/or an antibody response. In various embodiments, the efficacy of TIMP-MG at relieving one or more symptoms of MG and/or reducing the duration and severity of an inflammatory immune response to one or more MG antigens is determined from the assay of one or more biological samples from the subject. In various embodiments, the biological samples are selected from the group consisting whole-blood, peripheral blood, peripheral blood mononuclear cells (PBMCs), serum, plasma, urine, cerebrospinal fluid (CSF), stool, a tissue biopsy, and/or a bone-marrow biopsy.

Also contemplated is a composition comprising TIMP-MG as described herein for use in treating myasthenia gravis. In various embodiments, the disclosure provides for use of a composition comprising TIMP-MG as described herein in the preparation of a medicament for treating myasthenia gravis.

The process for manufacturing of TIMP-MG involves numerous steps each of which influences the physiochemical properties of resulting composition essential for safe and therapeutic administration. Importantly, the process must be optimized to ensure efficient encapsulation of MG antigens within the particle core.

In various embodiments, TIMP-MG encapsulates one or more MG associated antigens or antigenic epitopes. In various embodiments, the antigen is the AChR protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3) and/or a portion thereof, or one or more antigenic epitopes thereof selected from table 1. In various embodiments, the antigen is the AChR protein subunit and/or a portion thereof. In various embodiments, the antigen is the AChR antigen and/or a portion thereof, or one or more antigenic epitopes thereof selected from the group consisting of SEQ ID NOS: 1 to 21 (Table 2). In various embodiments, TIMP-MG encapsulates antigens comprising a combination of SEQ ID NO: 3, SEQ ID NO: 5, SEQ ID NO: 8, SEQ ID NO: 13, SEQ ID NO: 19, SEQ ID NO: 20, and/or SEQ ID NO: 21 (Table 2).

The present disclosure provides a process for manufacturing a composition comprising negatively charged particles encapsulating one or more MG associated antigens (TIMP-MG). The process is directed to a process of manufacturing particles optimized for safe and therapeutic administration of TIMP-MG for the treatment of MG. In various embodiments, the method comprises: (a) generating an aqueous solution of one or more MG associated antigens; (b) generating a primary emulsion by mixing an aqueous solution of one or more MG associated antigens with an oil phase including a polymer; (c) mixing the primary emulsion with a solution including one or more surfactants and/or stabilizers to form a secondary emulsion; (d) hardening the secondary emulsion by evaporation resulting in hardened polymeric nanoparticles encapsulating MG associated antigens within their cores.

In various embodiments, the method further comprises the step (e) filtering, washing, and concentrating the nanoparticles. In various embodiments, the method further comprises the step (e) filtering, washing, and concentrating the nanoparticles and (f) freeze drying the nanoparticles. In various embodiments, the primary emulsion of step (b) is a water-in-oil emulsion. In various embodiments, the secondary emulsion of step (c) is an oil-in-water emulsion.

In various embodiments the one or more MG associated antigens in step (a) are dissolved in an aqueous solution comprising a solvent. In various embodiments the solvent comprises an organic solvent. In various embodiments the solvent comprises an inorganic solvent. In various embodiments the one or more MG associated antigens are dissolved in a solvent that comprises one or more acids and/or one or more base. In various embodiments the solvent has a pH between pH 1.0 and pH 14.0. In various embodiments the pH is about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, or about 14.0 including all values lying within this range.

In various embodiments the solvent comprising acid is selected from the group comprising acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic acid, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid. In various embodiments the solvent comprising base is selected from the group comprising barium hydroxide, calcium hydroxide, chromium hydroxide, potassium hydroxide, ammonium hydroxide, zinc hydroxide, barium hydroxide, sodium bicarbonate, methylamine, diethylamine, sodium hydroxide, magnesium hydroxide, ammonium bicarbonate, ammonia, aluminium hydroxide, sodium carbonate, magnesium hydroxide, zinc hydroxide, ferrous hydroxide, acetone, lithium hydroxide, pyridine, rubidium hydroxide. In various embodiments the concentration of the acid is between 0.1 N to 36 N. In various embodiments the acid solvent concentration is about 0.1N, about 0.5N, about 1N, about 2N, about 3N, about 4N, about 5N, about 6N, about 7N, about 8N, about 9N, about 10N, about 11N, about 12N, about 13N, about 14N, about 15N, about 16N, about 17N, about 18N, about 20N, about 30N, or about 36N including all values lying within this range. In various embodiments, the concentration of the base solvent is about 0.01%, about 0.05%, about 0.1%, about 0.2%, about 0.3%, about 0.4%, about 0.5%, about 0.6%, about 0.7%, about 0.8%, about 0.9% about 1%, about 10%, about 25%, about 50%, about 75%, about 100% (v/v or wt/v) including all values lying within this range.

In various embodiments, the one or more MG associated antigens in step (a) are dissolved in an aqueous solution comprising one or more stabilizers. In various embodiments, the stabilizers comprise detergents/surfactants, osmolytes, metal complexes, proteins or amino acids. In various embodiments, the stabilizers are selected from the group comprising poly vinyl alcohol, sorbitan monostearate, TritonX, Triton X-100, poloxamer, polyvinylpyrrolidone, Pluronics F68, n-dodecyl-β-D-maltoside (DDM), lauryl maltose neopentyl glycol (LMNG), 1-myristoyl-2-hydroxy-sn-glycero-3-[phospho-rac-(1-glycerol)] (LMPG), 1-palmitoyl-2-hydroxy-sn-glycero-3-[phospho-rac-(1-glycerol)] (LPPG), polyethylene glycol 400 dedecyl ether (Thesit), nonylphenyl polyethylene glycol (NP40), polyoxyethylene-(10)-dodecyl-ether (Genapol C-100), dodecyl-phosphocholine (DPC), n-decyl-β-maltoside (DM), 1,2-dioctanoyl-sn-glycero-3-phosphocholine (diC₈PC), 1,2-dihexanoyl-sn-glycero-3-phosphocholine (diC₆PC), 1,2-diheptanoyl-sn-glycero-3-phosphocholine (DHPC), n-octyl-β-D-glucose (B-OG), Brih-35, Brij-56, Brij-58, Brij-72, Brij-78, Brij-97, Brij-98, 3-((3-cholamidopropyl)dimethylammonio)-1-propanesulfonate) (CHAPS), disulphile containing detergents, fluorinated diglucose detergents, fluorinated maltose detergents, 1,3,5-triazine-cored maltoside amphiphiles, steroid based pentasaccharides, Vitamin-E based glycoside amphiphiles, calixarene based detergents, cyclodextrin, trehalose, mannitol, dextran, carboxymethyl cellulose, ethyl stearate, sodium acetate, sodium glutamate, polysorbate, albumin, bovine serum albumin, lysine, histidine, arginine, zinc salts, sodium bicarbonate, or magnesium hydroxide. In various embodiments the concentration of the stabilizer in step (a) is between 0.01% and 15% (e.g. about 0.01%, 0.05%, 0.1%, 0.5%, 1%, 5%, 10% or 15%) including all values lying within this range.

In various embodiments, the concentration of one or more MG associated antigens dissolved in step (a) is between 0.1 mg/mL and 100 mg/mL. In various embodiments, the concentration of one or more MG associated antigens dissolved in step (a) is about 0.1 mg/mL, about 0.2 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL including all values lying within this range.

In various embodiments, the one or more MG-associated antigens are dissolved in the solvent by mixing for about 0.1, about 0.2, about 0.3, about 0.4, about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 48, about 72, or about 96 hours including all values lying within this range.

In various embodiments, the one or more MG associated antigens are manufactured synthetically. In various embodiments, the antigens are manufactured by solid phase peptide synthesis or solution phase peptide synthesis. In various embodiments, the one or more MG associated antigens are manufactured using recombinant protein production.

In various embodiments, one or more MG associated antigens in step (a) are dissolved in an aqueous solution comprising a solvent. In various embodiments, one or more MG associated antigens in step (a) are dissolved in the same solvent. In various embodiments the one or more MG associated antigens in step (a) are dissolved in different solvents. One or more MG associated antigens in step (a) are dissolved in a solvent additionally comprising one or more stabilizers. Depending on their solubility and/or stability in acid or base, one or more antigens comprised within TIMP-MG are dissolved in an acid whereas others are dissolved in a base. In various embodiments antigens dissolved in acid and antigens dissolved in a base are admixed prior to, during or after generation of the primary emulsion in step (b). In various embodiments, the antigens are admixed at a ratio of 100:1, 50:1, 25:1, 20:1, 10:1, 9:1, 8:1, 7:1, 6:1, 5:1, 4:1, 3:1, 2:1 or 1:1 including all values lying within this range. In some embodiments, MG associated antigens comprising SEQ ID NO: 3, SEQ ID NO: 8, SEQ ID NO: 13, SEQ ID NO: 19, SEQ ID NO: 20, SEQ ID NO: 21 (Table 2) are dissolved in a acid. In some embodiments, MG associated antigen comprising SEQ ID NO: 5 (Table 2) were dissolved in base. In some embodiments, acid dissolved MG associated antigen comprising SEQ ID NO: 3, SEQ ID NO: 8, SEQ ID NO: 13, SEQ ID NO: 19, SEQ ID NO: 20, SEQ ID NO: 21 (Table 2) are mixed with base dissolved MG associated antigen comprising SEQ ID NO: 5 (Table 2) at a ratio of 4:1.

In various embodiments, the MG associated antigen is an AChR protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3) and/or a portion thereof, or one or more antigenic epitopes thereof. In various embodiments, the MG associated antigen is an AChR protein subunit and/or a portion thereof, or one or more antigenic epitopes thereof. In various embodiments, the MG associated antigen is an AChR antigen and/or a portion thereof, or one or more antigenic epitopes thereof from table 1. In various embodiments, the MG associated antigen is an AChR antigen and/or a portion thereof, or one or more antigenic epitopes thereof selected from the group consisting of AChR antigens with SEQ ID NOS: 1 to 21 (Table 2). In various embodiments the MG associated antigens comprise a combination of SEQ ID NO: 3, SEQ ID NO: 5, SEQ ID NO: 8, SEQ ID NO: 13, SEQ ID NO: 19, SEQ ID NO:20, and/or SEQ ID NO: 21 (Table 2).

In various embodiments, the emulsion of step (b) includes a solvent. In various embodiments, the solvent is an organic solvent. In various embodiments, the solvent is an inorganic solvent. In various embodiments, the organic solvent is selected from the group comprising acetone, ethanol, methylene chloride (dichloromethane), dimethyl sulfoxide (DMSO), ethyl acetate, dimethylformamide, tetrahydrofuran, chloroform, and acetic acid. In various embodiments, the inorganic solvent is selected from the group comprising water, ammonia, sulphuric acid, carbon disulphide, bromine trifluoride, phosphorous oxychloride, hydrogen fluoride, and sulphur dioxide. In various embodiments, the solvent of step (b) is at a concentration between 1% (v/v) to 50% (v/v) including all values lying within this range. In various embodiments, the solvent of step (b) is at a concentration of about 1%, about 2%, about 3%, about 5%, about 10%, about 15%, about 20%, about 25%, about 30%, about 35%, about 40%, about 45%, or about 50% (v/v). In various embodiments, the solvent of step (b) is at a concentration between 0.1 mM and 10.0 mM including all values lying within this range. In various embodiments, the solvent of step (b) is at a concentration of 0.1 mM, 0.2 mM, 0.3 mM, 0.4 mM, 0.5 mM, 0.6 mM, 0.7 mM, 0.8 mM, 0.9 mM, 1.0 mM, 1.5 mM, 2 mM, 2.5 mM, 3 mM, 3.5 mM, 4 mM, 4.5 mM, 5 mM, 5.5 mM, 6 mM, 6.5 mM, 7.0 mM, 7.5 mM, 8.0 mM, 8.5 mM, 9.0 mM, or 10.0 mM. In various embodiments, the solvent of step (b) is at a concentration between 0.1 M and 10.0 M including all values lying within this range.

In various embodiments, the surfactant and/or stabilizer solution of step (c) includes a solvent. In various embodiments, the solvent is an organic solvent. In various embodiments, the solvent is an inorganic solvent. In various embodiments, the organic solvent is selected from the group comprising acetone, ethanol, methylene chloride (dichloromethane), dimethyl sulfoxide (DMSO), ethyl acetate, dimethylformamide, tetrahydrofuran, chloroform, and acetic acid. In various embodiments, the inorganic solvent is selected from the group comprising water, ammonia, sulphuric acid, carbon disulphide, bromine trifluoride, phosphorous oxychloride, hydrogen fluoride, and sulphur dioxide. In various embodiments, the solvent in the mixture is at a concentration between 1% and 50% (v/v) including all values lying within this range. In various embodiments, the solvent in the mixture is at a concentration of 1%, 2%, 3%, 5%, 10%, 15%, 20%, 25%, 30%, 35%, 40%, 45%, or 50% (v/v). In various embodiments, the solvent in the mixture is at a concentration between 0.1 mM and 10.0 mM including all values lying within this range. In various embodiments, the solvent in the mixture is at a concentration of 0.1 mM, 0.2 mM, 0.3 mM, 0.4 mM, 0.5 mM, 0.6 mM, 0.7 mM, 0.8 mM, 0.9 mM, 1.0 mM, 1.5 mM, 2 mM, 2.5 mM, 3 mM, 3.5 mM, 4 mM, 4.5 mM, 5 mM, 5.5 mM, 6 mM, 6.5 mM, 7.0 mM, 7.5 mM, 8.0 mM, 8.5 mM, 9.0 mM, or 10.0 mM. In various embodiments, the solvent in the mixture is at a concentration of 0.1M and 10.0 M including all values lying within this range. In various embodiments, the solvent in the mixture is at a concentration of 0.1M, 0.2M, 0.3M, 0.4M, 0.5M, 0.6M, 0.7M, 0.8M, 0.9M, 1.0M, 1.5M, 2M, 2.5M, 3M, 3.5M, 4M, 4.5M, 5M, 5.5M, 6M, 6.5M, 7.0M, 7.5M, 8.0M, 8.5M, 9.0M, or 10.0 M. In various embodiments, the solvent of step (b) and step (c) are the same. In various embodiments, the solvents of step (b) and step (c) are different.

In various embodiments, the polymer in step (b) is a biodegradable polymer. In various embodiments, the biodegradable polymer is polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid. In various embodiments, the polymer is a co-polymer. In various embodiments, the co-polymer has varying molar ratios of constituent polymers. In various embodiments, the molar ratio is 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0 including all values lying within this range.

In various embodiments, the polymer in step (b) is PLGA. In various embodiments, the molar ratio of co-polymers of PLGA are 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0 including all values lying within this range. In various embodiments, the PLGA has a high molecular weight. In various embodiments, the PLGA has a low molecular weight. In various embodiments, the PLGA has a molecular weight between 1 kDa and 100 kDa (e.g., between 1 kDa, 5 kDa, 10 kDa, 20 kDa, 30 kDa, 40 kDa, 50 kDa, 60 kDa, 70 kDa, 80 kDa, 90 kDa, 100 kDa including all values lying within this range). In various embodiments, the amount of PLGA in the solution of step (b) is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight. In various embodiments, the polymer in step (b) is dissolved in a solvent. In various embodiments, the polymer in step (b) is dissolved in a solvent selected from the group comprising acetone, ethyl formate, methylene chloride (dichloromethane), ethyl acetate, dimethylformamide, tetrahydrofuran, or chloroform. In various embodiments, the PLGA polymer is step (b) is dissolved in ethyl acetate. In various embodiments, the step (b) comprises 5% PLGA solution (50:50) having a molecular weight between 10,000 and 60,000 Daltons (Da).

In various embodiments, the surfactant and/or stabilizer used in step (c) is anionic, cationic, or nonionic. In various embodiments, the surfactant and/or stabilizer is selected from the group comprising poloxamer, a polyamine, polyethylene glycol (PEG), Tween-80, gelatin, dextran, pluronic L-63, pluronic F-68, pluronic 188, pluronic F-127, polyvinyl alcohol (PVA), polyacrylic acid (PAA), methylcellulose, lecithin, didodecyldimethylammonium bromide (DMAB), poly(ethylene-alt-maleic acid) (PEMA), vitamin E TPGS (D-a-tocopheryl polyethylene glycol 1000 succinate), hyaluronic acid, poly amino acids (e.g., polymers of lysine, arginine, aspartic acid, glutamic acid, serine, threonine, tyrosine and cysteine, or their enantiomers), methylcellulose, hydroxyethylcellulose, hydroxyprolylcellulose, hydroxypropylmethylcellulose, gelatin, sodium cholate, a carbomer, or a sulfate polymer (e.g., heparin sulfate, chondroitin sulfate, fucoidan, ulvan, and carrageenan). In various embodiments, the amount of surfactant and/or stabilizer present in step (c) is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the surfactant and/or stabilizer have a molecular weight between 0.1 and 10,000 kDa (e.g., between 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 500, 1000, 2000, 3000, 4000, 5000, 6000, 7000, 8000, 9000, or 10000 kDa including all values lying within this range). In various embodiments, the surfactant and stabilizer mixture is prepared by admixing 4% PVA and PAA (100 kDa, 35% wt) with ethyl acetate. In various embodiments, surfactant and stabilizer mixture is prepared by admixing 4% PVA in water and PAA (100 kDa, 35% wt) in water with ethyl acetate.

In various embodiments, the mixture including one or more surfactants and/or stabilizers that form an oil-in-water secondary emulsion in (c) has a pH less than 4.0. In various embodiments, the oil-in-water secondary emulsion has a pH of about pH 1 to about pH 4, about pH 2 to about pH 4, about pH 3 to about pH 4, or about pH 1, about pH 1.5, about pH 2, about pH 2.5, about pH 3, about pH 3.5, about pH 4 including all ranges and values that lie between these ranges.

In various embodiments, the method comprises: (a) generating an aqueous solution of one or more MG associated antigens; (b) generating a primary emulsion by mixing an aqueous solution of one or more MG associated antigens with an oil phase including a polymer; (c) mixing the primary emulsion with a solution including one or more surfactants and/or stabilizers to form a secondary emulsion; (d) hardening the secondary emulsion by evaporation to remove the solvent resulting in hardened polymeric nanoparticles encapsulating MG associated antigens within their cores; (e) filtering, washing, and concentrating the nanoparticles; and (f) freeze drying the nanoparticles.

In various embodiments, the water-in-oil primary emulsion of step (b) is obtained by homogenization of the aqueous solution of one or more MG associated antigens with the oil phase including a polymer. In various embodiments, homogenization of step (b) is performed between 5 seconds and 1000 seconds including all values lying within this range. In various embodiments, homogenization is performed for 5, 10, 15, 20, 25, 30, 40, 45, 60, 90, 120, 150, 180, 210, 240, 270, 300, 330, 360, 390, 420, 450, 480, 510, 540, 570, 600, 700, 800, 900, or 1000 seconds. In various embodiments, the oil-in-water secondary emulsion of step (c) is obtained by homogenization of the primary emulsion with a solution including one or more surfactants and/or stabilizer. In various embodiments, homogenization of step (c) is performed between 5 seconds and 1000 seconds including all values lying within this range. In various embodiments, homogenization is performed for 5, 10, 15, 20, 25, 30, 30, 40, 45, 60, 90, 120, 150, 180, 210, 240, 270, 300, 330, 360, 390, 420, 450, 480, 510, 540, 570, 600, 700, 800, 900, or 1000 seconds. In various embodiments, the water-in-oil primary emulsion of step (b) is obtained by sonication of the aqueous solution of MG associated antigens with the oil phase including a polymer. In various embodiments, sonication in step (b) is performed between 5 and 1000 seconds including all values lying within this range. In various embodiments, sonication in step (b) is performed for 5, 10, 15, 20, 25, 30, 30, 40, 45, 60, 90, 120, 150, 180, 210, 240, 270, 300, 330, 360, 390, 420, 450, 480, 510, 540, 570, 600, 700, 800, 900, or 1000 seconds. In various embodiments, the oil-in-water secondary emulsion of step (c) is obtained by sonication of the primary emulsion with a solution including one or more surfactants and/or stabilizers. In various embodiments, sonication in step (c) is performed between 5 and 1000 seconds including all values lying within this range. In various embodiments, sonication in step (c) is performed for 5, 10, 15, 20, 25, 30, 30, 40, 45, 60, 90, 120, 150, 180, 210, 240, 270, 300, 330, 360, 390, 420, 450, 480, 510, 540, 570, 600, 700, 800, 900, or 1000 seconds.

In various embodiments, the secondary emulsion is hardened by evaporation. In various embodiments, the evaporation is active evaporation. In various embodiments, the active evaporation is performed using stirring or under vacuum. In various embodiments, the active evaporation is performed under high-pressure vacuum. In various embodiments, the active evaporation is performed under low pressure vacuum. In various embodiments, the evaporation is passive evaporation. In various embodiments, the evaporation is performed between 0.25 and 96 hours including all values lying within this range. In various embodiments, the evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours. In various embodiments, the evaporation is performed at a pressure of between 0.01 and 1000 mBar (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 mBar including all including all values lying within this range).

In various embodiments, the filtration, washing, and concentration of particles in step (c) is performed by gel filtration, membrane filtration, dialysis, centrifugation, chromatography, density gradient centrifugation, or combinations thereof.

In various embodiments, the present disclosure further contemplates a method for manufacturing a composition comprising negatively charged particles encapsulating a combination of MG associated antigens comprising SEQ ID NO: 3, SEQ ID NO: 5, SEQ ID NO: 8, SEQ ID NO: 13, SEQ ID NO: 19, SEQ ID NO: 20, SEQ ID NO: 21 listed in Table 2 comprising: (a) dissolving peptides of SEQ ID NO: 3, SEQ ID NO: 8, SEQ ID NO: 13, SEQ ID NO 19, SEQ ID NO 20 and SEQ ID NO: 21 in acetic acid, and dissolving peptide of SEQ ID NO: 5 in ammonium hydroxide followed by admixing the acid dissolved peptides with base dissolved peptide at a 4:1 ratio; (b) generating a primary emulsion by homogenizing the aqueous solution of (a) with an oil phase including PLGA dissolved in ethyl acetate; (c) homogenizing the primary emulsion with a mixture comprising ethyl acetate, polyacrylic acid (PAA) and polyvinyl alcohol (PVA) to form a secondary emulsion; (d) hardening the secondary emulsion by evaporation resulting in hardened polymeric nanoparticles encapsulating peptides of SEQ ID NO: 3, SEQ ID NO: 5, SEQ ID NO: 8, SEQ ID NO: 13, SEQ ID NO: 19, SEQ ID NO: 20, SEQ ID NO: 21 within their cores.

In various embodiments, the method further comprises (e) filtering, washing, and concentrating the nanoparticles. In various embodiments, the method further comprises (e) filtering, washing, and concentrating the nanoparticles and (f) addition of sodium citrate, mannitol, and sucrose, and freeze drying the particles.

In various embodiments, the disclosure also contemplates a method for manufacturing a composition comprising negatively charged particles encapsulating a combination of MG associated antigens comprising SEQ ID NO: 3, SEQ ID NO: 5, SEQ ID NO: 8, SEQ ID NO: 13, SEQ ID NO: 19, SEQ ID NO: 20, SEQ ID NO: 21 listed in Table 2 comprising: (a) dissolving peptides of SEQ ID NO: 3, SEQ ID NO: 8, SEQ ID NO: 13, SEQ ID NO: 19, SEQ ID NO: 20 and SEQ ID NO: 21 in acetic acid, and dissolving peptide of SEQ ID NO: 5 in ammonium hydroxide followed by admixing the acid dissolved peptides with base dissolved peptide at a 4:1 ratio; (b) generating a primary emulsion by homogenizing the aqueous solution of (a) with an oil phase including PLGA dissolved in ethyl acetate; (c) homogenizing the primary emulsion with a mixture comprising ethyl acetate, polyacrylic acid (PAA) and polyvinyl alcohol (PVA) to form a secondary emulsion; (d) hardening the secondary emulsion by evaporation resulting in hardened polymeric nanoparticles encapsulating peptides of SEQ ID NO: 3, SEQ ID NO:ID NO:: 5, SEQ ID NO: 8, SEQ ID NO: 13, SEQ ID NO: 19, SEQ ID NO: 20, and/or SEQ ID NO: 21 within their cores; (e) filtering, washing, and concentrating the nanoparticles; and (f) addition of sodium citrate, mannitol, and sucrose, and freeze drying the particles.

In various embodiments, the acetic acid concentration in step (a) is between 0.1 N to 1 N including all values lying within this range. In various embodiments, the acetic acid concentration in step (a) is 0.5 N.

In various embodiments, the ammonium hydroxide concentration in step (b) is between 0.01% to 1% including all values lying within this range. In various embodiments, the ammonium hydroxide concentration in step (b) is 0.1%.

In various embodiments, the method comprises a 5% PLGA solution (50:50) with a molecular weight between 10,000 to 60,000 Da in step (b).

In various embodiments, the mixture of (c) is prepared by adding 4% PVA and PAA (100 kDa, 35% wt) to ethyl acetate. In various embodiments, the PVA/PAA/ethyl acetate mixture is maintained at a pH below pH 4.0, below pH 3.9, below pH 3.8, below pH 3.7, below pH 3.6, or below pH 3.5.

The present disclosure also contemplates a process for manufacturing a composition comprising negatively charged TIMPs encapsulating one or more MG associated antigens (TIMP-MG) wherein the TIMP-MG particles have a negative zeta potential. In various embodiments, the negative zeta potential of TIMP-MG particles manufactured by the process is between about −100 mV to about 0 mV. In various embodiments, the zeta potential of the particles is from about −100 mV to about −25 mV, from about −100 to about −30 mV, from about-80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV.

The present disclosure contemplates a process for manufacturing a composition comprising negatively charged TIMPs encapsulating one or more MG associated antigens (TIMP-MG) wherein the size or diameter of the particles is between 0.05 μm to about 10 μm. In various embodiments, the diameter of TIMP-MG particles is between 0.1 μm and about 10 μm. In various embodiments, the diameter of TIMP-MG particles is between 0.1 μm and about 5 μm. In various embodiments, the diameter of TIMP-MG particles is between 0.1 μm and about 3 μm. In various embodiments, the diameter of TIMP-MG particles is between 0.3 μm and about 5 μm. In various embodiments, the diameter of TIMP-MG particles is about 0.3 μm to about 3 μm. In various embodiments, the diameter of TIMP-MG particles is between about 0.3 μm to about 1 μm. In various embodiments, the diameter of TIMP-MG particles is between about 0.4 μm to about 1 μm. In various embodiments, the TIMP-MG particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the diameter of the negatively charged particle is between 400 nm to 800 nm. In various embodiments, the polydispersity index (PDI) or heterogeneity index for particle size is between 0.01 and 1.0 (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 including all values within the range).

Also contemplated is a process for manufacturing a composition comprising negatively charged TIMPs encapsulating one or more MG associated antigens (TIMP-MG) wherein the particles have a homogenous size distribution. In various embodiments, the particles have a homogenous size distribution wherein at least 90% of the particles have a diameter of between 0.05 μm and about 10 μm, between 0.1 μm and about 10 μm, 0.1 μm and about 5 μm, 0.1 μm and about 3 μm, 0.3 μm to about 2 μm, 0.3 μm and about 5 μm, 0.3 μm to about 3 μm, 0.3 μm to about 1 μm, or 0.4 μm to about 1 μm. In various embodiments, the particles have a homogenous size distribution wherein at least 90% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 50% of the particles have a diameter of between about 0.05 μm and about 10 μm, about 0.1 μm and about 10 μm, about 0.1 μm and about 5 μm, about 0.1 μm and about 3 μm, 0.3 μm to about 2 μm, about 0.3 μm and about 5 μm, and about 0.3 μm and about 3 μm, 0.3 μm to about 1 μm, or 0.4 μm to about 1 μm. In various embodiments, the particles have a homogenous size distribution wherein at least 50% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 10% of the particles have a diameter of between about 0.05 μm and about 10 μm, about 0.1 μm and about 10 μm, about 0.1 μm and about 5 μm, about 0.1 μm and about 3 μm, 0.3 μm to about 2 μm, about 0.3 μm and about 5 μm, and about 0.3 μm and about 3 μm, 0.3 μm to about 1 μm, or 0.4 μm to about 1 μm. In various embodiments, the particles have a homogenous size distribution wherein at least 10% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-MG particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In embodiments, the negatively charged particles have a D90 of about less than 1000 nm. In embodiments, the negatively charged particles have a D50 of about 400 nm to about 800 nm, including about 400 nm, about 420 nm, about 440 nm, about 460 nm, about 480 nm, about 500 nm, about 510 nm, about 520 nm, about 530 nm, about 540 nm, about 550 nm, about 560 nm, about 570 nm, about 580 nm, about 590 nm, about 600 nm, about 650 nm, about 700 nm, about 750 nm, or about 800 nm including any values or ranges there between. In embodiments, the negatively charged particles have a D10 of about less than 600 nm. In embodiments, the negatively charged particles have a D90 of about 600 nm to about 700 nm. In embodiments, the negatively charged particles have a D50 of about 550 nm to about 600 nm. In embodiments, the negatively charged particles have a D10 of about 500 nm to about 550 nm. In various embodiments the negatively charged particles have a D [4,3] to less than 1000 nm.

In various embodiments, the invention of the present disclosure provides a process for manufacturing a composition comprising negatively charged particles encapsulating one or more MG associated antigens (TIMP-MG). In various embodiments, the amount of one or more MG associated antigens encapsulated within the TIMP-MG composition is between 0.1 μg/mg of polymer and 100 μg/mg of polymer including all values lying within this range. In various embodiments, the MG associated antigen content is between 0.1 μg/mg of PLGA to 100 μg/mg of PLGA (e.g., 0.1, 0.5, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, or 100 μg/mg) including all values and ranges that lie in between these values. In various embodiments, the process of making TIMP-MG as described herein yields an encapsulation efficiency between 1-100% (e.g., 1, 2, 3, 4, 5, 6, 7, 8, 9, 10, 15, 20, 25, 30, 40, 50, 60, 70, 80, 90, or 100% including all values and ranges that lie between these values). The one or more MG associated antigen content in the TIMP-MG composition can be determined by methods described in the literature including ELISA, Mass Spectrometry, HPLC, CBQCA, and Western Blot.

In various embodiments, the present disclosure provides a process for manufacturing a composition comprising negatively charged particles encapsulating one or more MG associated antigens (TIMP-MG), wherein the particle surface contains low levels of one or more MG associated antigens. In various embodiments, the particle surface is essentially free of one or more MG associated antigens. In various embodiments, the amount of one or more MG associated antigens present on the surface of the particles is between 0-30% (e.g., 0, 1, 2, 3, 4, 5, 6, 7, 8, 9, 10, 11, 12, 13, 14, 5, 16, 17, 18, 19, 20, 21, 22, 23, 24, 25, 26, 27, 28, 29, or 30% including all values and ranges that lie between these values) of the total antigen content of the TIMP-MG composition. In various embodiments, the frequency of particles containing one or more MG associated antigens on their surface is between 0-30% (e.g., 0, 1, 2, 3, 4, 5, 6, 7, 8, 9, 10, 11, 12, 13, 14, 5, 16, 17, 18, 19, 20, 21, 22, 23, 24, 25, 26, 27, 28, 29, or 30% including all values and ranges that lie between these values) higher compared to a negative control. In various embodiments, the frequency of particles containing one or more MG associated antigens on their surface is 0-100% % (e.g., 0, 1, 2, 3, 4, 5, 6, 7, 8, 9, 10, 20, 30, 40, 50, 60, 70, 80, 90, and 100% including all values and ranges that lie between these values) lower when compared to a positive control. In various embodiments, the amount of one or more MG associated antigens on the surface of the particles is between 0-10-fold (e.g., 0, 1, 2, 3, 4, 5, 6, 7, 8, 9, or 10 fold including all values and ranges that lie between these values) higher than a negative control. In various embodiments, the amount of one or more MG associated antigens on the surface of the particles is between 0-100-fold (e.g., 0, 1, 2, 3, 4, 5, 6, 7, 8, 9, 10, 20, 30, 40, 50, 60, 70, 80, 90, or 100-fold including all values and ranges that lie between these values) lower than a positive control. In various embodiments, the number of TIMP-MG particles with one or more MG associated antigens on their surface is determined using previously described methods such as flow cytometry, Mass Spectrometry, ELISA, CBQCA, and Western Blot.

In various embodiments, the present disclosure provides a process for manufacturing a composition comprising negatively charged particles encapsulating one or more MG associated antigens (TIMP-MG), wherein the particles exhibit low burst release. In various embodiments, the particles exhibit no burst release. In various embodiments, the particle burst release is between 0-85% (e.g., 0, 1, 2, 3, 4, 5, 6, 7, 8, 9, 10, 20, 30, 40, 50, 60, 70, 75, 80, or 85% including all values and ranges that lie between these values).

In various embodiments, excipients are added to the nanoparticle composition prior to freeze drying in step (f). In various embodiments, the excipients are buffering agents and/or cryoprotectants. In various embodiments, the excipients are selected from the group consisting of sucrose, mannitol, trehalose, sorbitol, dextran, Ficoll, Dextran 70k, sodium citrate, lactose, glycol, glycerol, cyclodextrin, L-arginine, or glycine. In various embodiments, the amounts of excipients added to the nanoparticle composition prior to freeze drying is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the amounts of excipients added to the nanoparticle composition prior to freeze drying is between 0.01 and 500 g (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, or 500 g) per gram of nanoparticles.

In various embodiments, the manufacturing batch sizes of TIMP-MG can be scaled up or down. In various embodiments, the manufacturing batch size is between 0.01 g to 100 kg. In various embodiments, the batch size is 0.01 g, 0.1 g, 10 g, 20 g, 40 g, 60 g, 80 g, 100 g, 160 g, 240 g, 320 g, 400 g, 480 g, 560 g, 640 g, 720 g, 800 g, 1000 g, 5 kg, 10 kg, 50 kg or 100 kg including all values and ranges that lie between these values.

Also provided is a composition comprising particles encapsulating MG associated antigens made by the methods described herein. In various embodiments, the composition further comprises a pharmaceutically acceptable carrier, diluent, or excipient. In various embodiments, the pharmaceutical composition is a sterile pharmaceutical composition.

In various embodiments, formulations or pharmaceutical compositions of TIMP-MG contain negatively charged particles encapsulating MG-associated antigens, and excipients. In various embodiments, the excipients are selected from the group consisting of sucrose, mannitol, trehalose, sorbitol, dextran, Ficoll, Dextran 70k, sodium citrate, lactose, L-arginine, or glycine. In various embodiments TIMP-MG formulations contain between one to eleven excipients. In various embodiments, TIMP-MG formulations contain one or more, two or more, three or more, four or more, five or more, six or more, seven or more, eight or more, nine or more, or ten or more excipients.

In various embodiments, TIMP-MG formulations contain negatively charged particles encapsulating MG associated antigens, sucrose, mannitol, and sodium citrate. In various embodiments, the negatively charged particle concentration in the TIMP-MG formulation is between 1 to 100% including all ranges and values that lie between these ranges. In various embodiments, the negatively charged particle concentration in the TIMP-MG formulation is between 20 to 50% including all ranges and values that lie between these ranges. In various embodiments, the negatively charged particle concentration in the TIMP-MG formulation is between 30 to 40% including all ranges and values that lie between these ranges. In various embodiments, the negatively charged particle concentration in the TIMP-MG formulation is about 30%, about 31%, about 32%, about 33%, about 34%, about 35%, about 35.6%, about 36%, about 37%, about 38%, about 39%, or about 40%.

In various embodiments, the sucrose concentration in the TIMP-MG formulation is between 1 to 100% including all ranges and values that lie between these ranges. In various embodiments, the sucrose in the TIMP-MG formulation is between 20 to 50% including all ranges and values that lie between these ranges. In various embodiments, the sucrose concentration in the TIMP-MG formulation is between 30 to 40% including all ranges and values that lie between these ranges. In various embodiments, the sucrose concentration in the TIMP-MG formulation is about 30%, about 31%, about 32%, about 33%, about 34%, about 35%, about 35.6%, about 36%, about 37%, about 38%, about 39%, or about 40%.

In various embodiments, the mannitol concentration in the TIMP-MG formulation is between 1 to 100% including all ranges and values that lie between these ranges. In various embodiments, the mannitol concentration in the TIMP-MG formulation is between 15 to 35% including all ranges and values that lie between these ranges. In various embodiments, the mannitol concentration in the TIMP-MG formulation is between 20 to 30% including all ranges and values that lie between these ranges. In various embodiments, the mannitol concentration in the TIMP-MG formulation is about 20%, about 21%, about 22%, about 23%, about 24%, about 25%, about 25%, about 26%, about 26.7%, about 27%, about 28%, about 29%, or about 30%.

In various embodiments, the sodium citrate concentration is between 0.01 to 25% including all ranges and values that lie between these ranges. In various embodiments, the sodium citrate concentration is between 0.5 to 3.5% including all ranges and values that lie between these ranges. In various embodiments, the sodium citrate concentration is about 0.5%, about 1%, about 1.5%, about 2%, about 2.1%, about 2.5%, about 3%, or about 3.5%.

In various embodiments, the total MG-associated antigen content in the TIMP-MG formulation is between 0.1 μg MG-associated antigens per mg of PLGA and 200 μg MG-associated antigens per mg of PLGA including all ranges and values that lie between these ranges. In various embodiments, the total MG-associated antigens or individual MG-associated antigen in the TIMP-MG formulation is between 2.1 μg antigens per mg of PLGA and 15.7 μg antigens per mg of PLGA including all ranges and values that lie between these ranges. In various embodiments, the individual MG-associated antigen in the TIMP-MG formulation is between 0.01 μg antigen per mg of PLGA and 50 μg antigen per mg of PLGA including all ranges and values that lie between these ranges. In various embodiments, an individual MG associated antigen in the TIMP-MG formulation is between 0.1 μg antigen per mg of PLGA and 5 μg antigen per mg of PLGA including all ranges and values that lie between these ranges. In various embodiments, the individual MG associated antigen in the TIMP-MG formulation is about 0.5 μg antigen per mg of PLGA, about 0.7 μg antigen per mg of PLGA, about 1 μg antigen per mg of PLGA, about 1.2 μg antigen per mg of PLGA, about 1.4 μg MG associated antigens per mg of PLGA, or about 1.8 μg MG associated antigens per mg of PLGA.

In various embodiments, the AChRα isoform a (324-357) (SEQ ID 3) antigen in the TIMP-MG formulation is between 0.05 μg/mg PLGA and 25 μg/mg PLGA. In various embodiments, the AChRε (116-130) (SEQ ID NO: 5) antigen in the TIMP-MG formulation is between 0.03 μg/mg PLGA and 23 μg/mg PLGA. In various embodiments, the AChRε (414-435) (SEQ ID NO: 8) antigen in the TIMP-MG formulation is between 0.01 μg/mg PLGA and 17 μg/mg PLGA. In various embodiments, the AChRα (43-58) (SEQ ID NO: 13) antigen in the TIMP-MG formulation is between 0.02 μg/mg PLGA and 22 μg/mg PLGA. In various embodiments, the AChRα (68-113) (SEQ ID NO: 19) antigen in the TIMP-MG formulation is between 0.06 μg/mg PLGA and 26 μg/mg PLGA. In various embodiments, the AChRα (121-158) (SEQ ID NO: 20) antigen in the TIMP-MG formulation is between 0.01 μg/mg PLGA and 21 μg/mg PLGA. In various embodiments, the AChRε (201-250) (SEQ ID NO: 21) antigen in the TIMP-MG formulation is between 0.03 μg/mg PLGA and 23 μg/mg PLGA.

In various embodiments, the AChRα isoform a (324-357) (SEQ ID 3) antigen in the TIMP-MG formulation is between 0.5 μg μg/mg PLGA and 2.5 μg/mg PLGA. In various embodiments, the AChRε (116-130) (SEQ ID NO: 5) antigen in the TIMP-MG formulation is between 0.3 μg/mg PLGA and 2.3 μg/mg PLGA. In various embodiments, the AChRε (414-435) (SEQ ID NO: 8) antigen in the TIMP-MG formulation is between 0.1 μg/mg PLGA and 1.7 μg/mg PLGA. In various embodiments, the AChRα (43-58) (SEQ ID NO: 13) antigen in the TIMP-MG formulation is between 0.2 μg/mg PLGA and 2.2 μg/mg PLGA. In various embodiments, the AChRα (68-113) (SEQ ID NO: 19) antigen in the TIMP-MG formulation is between 0.6 μg/mg PLGA and 2.6 μg/mg PLGA. In various embodiments, the AChRα (121-158) (SEQ ID 20) antigen in the TIMP-MG formulation is between 0.1 μg/mg PLGA and 2.1 μg/mg PLGA. In various embodiments, the AChRε (201-250) (SEQ ID NO: 21) antigen in the TIMP-MG formulation is between 0.3 μg/mg PLGA and 2.3 μg/mg PLGA.

The disclosure provides for methods of treating MG in a subject comprising administering to the subject particles encapsulating MG associated antigens as described herein. Also contemplated is a composition comprising TIMP-MG as described herein for use in treating MG. In various embodiments, the disclosure provides for use of a composition comprising TIMP-MG as described herein in the preparation of a medicament for treating MG.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawing and/or claims.

### BRIEF DESCRIPTION OF THE FIGURES

FIG. 1A-1D: CNP-106 inhibits antigen-specific T cell responses in animal models. The efficacy of TIMP-MG (CNP-106) at inducing tolerance was examined in a therapeutic mouse model of MG. TIMP-MG consists of negatively charged PLGA particles with zeta potential between −30 to −80 mV with a size diameter between 400 nm to 800 nm encapsulating AChR antigens with sequence SEQ ID NO: 3, SEQ ID NO: 5, SEQ ID NO: 8, SEQ ID NO:ID NO:: 13, SEQ ID NO: 19, SEQ ID NO: 20, SEQ ID NO: 21 selected from table 2. CNP-106 treatment (1.25 mg/dose) significantly improved grip strength compared to the saline treated group (FIG. 1A). CNP-106 treated mice also showed a trend towards improvement in overall clinical score (FIG. 1B), and electromyography (EMG) scores (FIG. 1C) when compared to the immunized saline control mice, with no weight change between the different treatment groups (FIG. 1D). (* p<0.05, *** p<0.0005)

FIG. 2A-2C: CNP-106 treatment inhibits AChR specific T-cell responses. AChR-specific T cell response was assessed via ex vivo recall responses from the spleen. Treatment of mice immunized with 1.25 mg/dose of CNP-106 decreased the levels of IL-2 (FIG. 2A), IL-10 (FIG. 2B) and IFN-gamma (FIG. 2C). (* p<0.05)

FIG. 3: Delayed type hypersensitivity. C57BL/6 mice were primed with 100 μg (200 μL injection volume) of AChR-peptides emulsified in CFA on Day 0. On Day 0 and Day 7 post priming, mice were treated with CNP-106 administered via IV injection at a dose of 0.1 mg dose/house (0.4 mg/kg HED), 0.5 mg dose/mouse (2 mg/kg HED), 1.25 mg dose/mouse (5 mg/kg HED), 2.5 mg dose/mouse (10 mg/kg HED). On Day 14 post priming, the mice were challenged intradermally with 10 μg MG peptides (right ear, 10 μL injection volume) or Ovalbumin (left ear, 10 μL injection volume). The pinna thickness of each ear was measured immediately post-elicitation with MG peptides and OVA as well as 24 hours post elicitation. The change in pinna thickness (ΔT) for both ears of each mouse was calculated to assess the DTH response. CNP-106 significantly inhibited the DTH response compared to unloaded control particles at 0.5 mg dose/mouse (2 mg/kg HED), 1.25 mg dose/mouse (5 mg/kg HED), and 2.5 mg/dose (10 mg/kg HED) (FIG. 3). (**** p<0.00005)

FIG. 4: Schedule of events for the Phase IA/IIB MG clinical trial. Primary objectives of this study include to assess the safety and tolerability of CNP-106. Subjects will be assessed for safety, tolerability, PD and efficacy according to the schedule of events (FIG. 4). In the post-dosing and follow-up period, subjects will return to the clinic for immune safety labs, PD measurements, QMG and MGC assessments, assessment of AEs, and medication changes per the Schedule of Events, (FIG. 4).

FIG. 5: Patient selection criteria for CNP-106 treatment. PBMCs were obtained from 10 MG patients to determine if there was a correlation with AChR restricted HLA positivity. Since CNP-106 encapsulates AChR antigens, subjects with HLA-DRB1*03, HLA-DRB3*01, HLA-DQB1*02 status can be a criteria for inclusion in the CNP-106 clinical trial. 80% of patients in the study expressed an HLA restriction, HLA-DRB1*03, HLA-DRB3*01, HLA-DQB1*02 for AChR (FIG. 5). Selection criteria enables identification of patients likely to achieve efficacy with CNP-105. MG patients whose disease is driven by autoreactivity to the AChR antigens are eligible for treatment with CNP-106 and can be identified based on HLA haplotype in blood.

FIG. 6 and FIG. 7: Process for manufacturing CNP-106. CNP-106 was manufactured using a double-emulsion solvent evaporation process. A high-level manufacturing process flow diagram is shown in FIG. 6. The final TIMP-MG formulation was characterized to determine physiochemical properties such as particle diameter, zeta potential, total peptide content. The results of TIMP-MG characterization are provided in Table 3. TIMP-MG particles were examined by Scanning Electron Microscopy showing a homogenous composition of intact particles with smooth surfaces (FIG. 7).

### DETAILED DESCRIPTION

There is a need for therapeutics for addressing immune imbalance in MG leading to improved disease symptoms and improved outcomes without the risk of toxic side-effects. TIMPs are surface functionalized negatively charged particles made of biodegradable material encapsulating antigenic proteins or peptide epitopes associated with inflammatory conditions such as autoimmune disease and allergies. TIMPs are designed for targeted delivery of encapsulated proteins/peptides to antigen presenting cells (APCs) of the mononuclear phagocyte system resulting in APC mediated T cell reprogramming via non-inflammatory pathways.

In pre-clinical models of autoimmune diseases and allergies, TIMPs have demonstrated therapeutic efficacy at inducing T-cell tolerance to antigenic/allergenic proteins and peptides resulting in improved disease symptoms. TIMPs encapsulating one or more antigens implicated in or associated with myasthenia gravis (TIMP-MG) can potentially treat MG by reprogramming the immune system and inducing antigen-specific T cell tolerance to MG associated antigens. There is a current need for immune tolerizing therapies which can induce tolerance to autoimmune MG associated antigens for long term therapeutic benefit without exposing patients to risk of adverse events.

The present disclosure provides compositions of negatively charged particles encapsulating one or more MG associated antigens, portions, or combinations thereof (TIMP-MG). Also included are methods of inducing antigen specific tolerance using TIMP-MG particles described herein, a process for manufacturing TIMP-MG particles encapsulating MG antigens and pharmaceutical compositions comprising the particles. The present disclosure provides methodology for monitoring the induction of and maintenance of immunologic tolerance in a subject having MG after receiving immunotherapy.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “tolerizing immune modifying particle”, the term “carrier particle”, and the term “bead” may be used interchangeably depending on the context. Additionally, the term “particle” may be used to encompass beads and spheres.

“Negatively charged particle” as used herein refers to particles which have been modified to possess a net surface charge that is less than zero.

“Surface-functionalized” as used herein refers to particles which have one or more functional groups on its surface. In some embodiments, the surface functionalization occurs by the introduction of one or more functional groups to a surface of a particle. In various embodiments, surface functionalization may be achieved by carboxylation (i.e., addition of one or more carboxyl groups to the particle surface) or addition of other chemical groups (e.g., other chemical groups that impart a negative surface charge).

“Carboxylated particles” or “carboxylated beads” or “carboxylated spheres” includes any particle that has been modified to contain a carboxyl group on its surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups, including, but not limited to, poly(ethylene-maleic anhydride) (PEMA).

As used herein, the term “Th cell” or “helper T cell” refers to CD4+ cells. CD4+ T cells assist other white blood cells with immunologic processes, including maturation of B cells into plasma cells and memory B cells, and activation of cytotoxic T cells and macrophages. T cells become activated when they are presented with peptide antigens by MHC class II molecules, which are expressed on the surface of antigen-presenting cells (APCs).

As used herein, the term “Th1 cell” refers to a subset of Th cells which produce pro-inflammatory mediators. Th1 cells secrete cytokines to facilitate immune response and play a role in host defense against pathogens in part by mediating the recruitment of neutrophils and macrophages to infected tissues. Th1 cells secrete cytokines including IFN-gamma, IL-2, IL-10, and TNF alpha/beta to coordinate defense against intracellular pathogens such as viruses and some bacteria.

As used herein, the term “Th2 cell” refers to a subset of Th cells that mediate the activation and maintenance of the antibody-mediated immune response against extracellular parasites, bacteria, allergens, and toxins. Th2 cells mediate these functions by producing various cytokines such as IL-4, IL-5, IL-6, IL-9, IL-13, and IL-17E (IL-25) that are responsible for antibody production, eosinophil activation, and inhibition of several macrophage functions, thus providing phagocyte-independent protective responses.

As used herein, the term ‘Th17 cell’ refers to a subset of Th cells that produce pro-inflammatory responses against extracellular bacteria and fungi. Th17 cells mediate these functions by producing cytokines such as IFN-gamma, IL-17A, IL-17F, IL-21, IL-22, TNF-alpha, and GM-CSF that are responsible for neutrophil, myeloid cell and B-cell recruitment.

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include post-expression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally-occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising via hosts cells that produce the proteins or through errors due to PCR amplification prior to host cell transfection.

“Antigenic moiety” or “antigen” as used herein refers to any moiety, for example a peptide, that is recognized by the host's immune system. Examples of antigenic moieties include, but are not limited to, autoantigens, allergens, enzymes, and/or bacterial or viral proteins, peptides, drugs or components.

“MG associated antigen” as used herein refers to an antigen, or a portion or fragment thereof, that results in an immune reaction against the protein or portion or fragment thereof. An MG associated antigen can include the whole protein or other protein or moiety associated with the protein that may elicit an immune response in a subject.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration) or via inhalation.

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three-dimensional structure that is identical to the epitope used to generate the antibody, yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure of the epitope and mimotope.

The term “therapeutically effective amount” is used herein to indicate the amount of antigen-specific composition of the disclosure that is effective to ameliorate or lessen one or more symptoms or signs of disease to be treated.

The term “symptom” is used herein to mean any physical or observable manifestation of a disorder, whether it is generally characteristic of that disorder or not. The term “symptoms” can mean all such manifestations or any subset thereof.

The terms “treat”, “treated”, “treating” and “treatment”, as used with respect to methods herein refer to eliminating, reducing, suppressing or ameliorating, either temporarily or permanently, either partially or completely, a clinical symptom, manifestation or progression of an event, disease or condition. Such treating need not be absolute to be useful.

### Particles

Tolerizing Immune Modifying Particles (TIMPs), comprising one or more antigens, have been previously described for the induction of antigen specific tolerance for treating inflammatory conditions (e.g., autoimmune diseases and allergies) (WO20131319253 and WO2015023796 incorporated herein by reference). In several preclinical models of autoimmune diseases and allergies, TIMPs have demonstrated efficacy at inducing antigen specific tolerance and inhibition of pathologic inflammatory immune responses.

The size and charge of the particles are important for tolerance induction. While the particles will differ in size and charge based on the antigen encapsulated within them, in general, particles described herein are effective at inducing tolerance when they are between about 100 nanometers and about 1500 nanometers and have a charge of between 0 to about −100 mV. In various embodiments, the particles are 400-800 nanometers in diameter and have a charge of between about −25 mV and −70 mV. In various embodiments, the particles are 400-800 nanometers in diameter and have a charge of between about −30 mV and −80 mV. In various embodiments, the particles are 400-800 nanometers in diameter and have a charge of between about −30 mV and −60 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PLGA particles, PLURONICS stabilized polypropylene sulfide particles, and diamond particles.

In some embodiments, the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS® (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles disclosed herein may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of citrate, glass, silica, polyesters of hydroxy carboxylic acids, polyanhydrides of dicarboxylic acids, or copolymers of hydroxy carboxylic acids and dicarboxylic acids. More generally, the TIMP-MG particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, TIMP-MG particles can be quantum dots, or composed of quantum dots, such as quantum dot polystyrene particles (Joumaa et al. (2006) Langmuir 22:1810-6). TIMP-MG particles including mixtures of ester and anhydride bonds (e.g., copolymers of glycolic and sebacic acid) may also be employed. For example, TIMP-MG particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), poly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the TIMP-MG particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

In certain embodiments, the particle is a co-polymer having a molar ratio from about 80:20 to about 100:0. Suitable co-polymer ratio of present immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In various embodiments, the particle is a PLURONICS stabilized polypropylene sulfide particle, a polyglycolic acid particle (PGA), a polylactic acid particle (PLA), or a poly(lactic-co-glycolic acid) particle. In various embodiments, the particle is a carboxylated PLGA particle. In various embodiments, the particles have a copolymer ratio of polylactic acid/polyglycolic acid 80:20:polylactic acid/polyglycolic acid 90:10: or polylactic acid:polyglycolic acid/50:50. In various embodiments, the particle is a poly(lactic-co-glycolic acid) particle and has a copolymer ratio of about 50:50 polylactic acid:polyglycolic acid. In various embodiments, the particle comprises about 50:50, about 80:20 to about 100:0 polylactic acid:polyglycolic acid or from about 50:50, about 80:20 to about 100:0 polyglycolic acid:polylactic acid. In various embodiments, the particle comprises 50:50 polylactic acid:polyglycolic acid. In various embodiments, the particle comprises polylactic acid:polyglycolic acid from about 99:1 to about 1:99, e.g., about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, and about 1:99, including all values and ranges that lie in between these values.

It is contemplated that the particle may further comprise a surfactant and/or stabilizer. The surfactant can be anionic, cationic, or nonionic. Surfactants in the poloxamer and poloaxamines family are commonly used in particle synthesis. Surfactants that may be used, include, but are not limited to PEG, Tween-80, gelatin, dextran, pluronic L-63, PVA, PAA, methylcellulose, lecithin, DMAB and PEMA. Additionally, biodegradable and biocompatible surfactants including, but not limited to, vitamin E TPGS (D-a-tocopheryl polyethylene glycol 1000 succinate), poly amino acids (e.g polymers of lysine, arginine, aspartic acid, glutamic acid, serine, threonine, tyrosine and cysteine, or their enantiomers), sodium cholate, and sulfate polymers. In some embodiments, two surfactants are used. In certain embodiments, two stabilizers are used. In certain embodiments, a combination of two or more surfactants and stabilizers are used. For example, if the particle is produced by a double emulsion method, the two surfactants can include a hydrophobic surfactant for the first emulsion, and a hydrophobic surfactant for the second emulsion. For example, stabilizers can be compounds which stabilize the primary and/or the secondary emulsion as described herein by providing a physical barrier or an energy barrier between adjacent nanoparticle droplets in the emulsion, thereby reducing their probability to coalesce and form larger nanoparticle droplets.

In various embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to reduce the nanoparticle size. One strategy to accomplish this is to apply a second strong shear rate. The leakage effect can be reduced by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference^{14,15}.

In some embodiments, the particle is a liposome. Liposomes may be prepared from a variety of lipid materials including, but not limited to, lipids of phosphatidyl choline, phosphatidyl serine, phosphatidyl inositol, phosphatidyl glycerol, phosphatidyl ethanolamine, phosphatidic acid, dicetyl phosphate, monosialoganglioside, polyethylene glycol, stearyl armine, ovolecithin and cholesterol, as well as mixtures of these in varying stoichiometries. Liposomes, as used herein, may also be formed from non-lipid amphipathic molecules, such as block copolymers of poly(oxyethylene-b-isoprene-b-oxye-thylene) and the like. In preferred embodiments, the liposomes are prepared from lipids or incorporate lipids that will form negatively charged liposomes, such as those produced from phosphatidyl serine, dicetyl phosphate, and dimyristoyl phosphatidic acid. In various embodiments, the negatively charged liposomes have a zeta potential from about −100 mV to about 0 mV, from about −100 mV to about −25 mV, from about −100 to about −30 mV, from about −80 mV to about −30 mV, from about −75 mV to about −30 mV, from about −70 mV to about −30 mV, from about −75 to about-35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and ranges therein. In various embodiments, the liposomes have a negative zeta potential of between −30 mV to −80 mV. In various embodiments, the liposomes have a negative zeta potential of between −30 mV to −60 mV. In various embodiments, the liposomes encapsulate one or more MG associated antigens, portions, or combinations thereof. In various embodiments, the antigen comprises one or more proteins, peptides, antigenic epitopes, or combinations thereof. In various embodiments, the TIMP-MG particles encapsulate one or more polynucleotides encoding MG associated antigens. In some embodiments, the polynucleotides comprise DNA, RNA, messenger RNA (mRNA), or circular RNA. In various embodiments, the antigen is acetylcholine receptor (AChR) protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3), portions thereof, and/or one or more antigenic epitopes thereof. In various embodiments, the one or more MG associated antigens are selected from the listing in Table 1. In various embodiments, the antigen is the AChR protein subunit and/or a portion thereof. In various embodiments, the antigen is the AChR antigen and/or a portion thereof, or one or more antigenic epitopes thereof selected from the group consisting of SEQ ID NOS: 1 to 21 (Table 2). In various embodiments, the one or more antigens encapsulated within the liposome comprises a combination of SEQ ID NO:3, SEQ ID NO:5, SEQ ID NO 8, SEQ ID NO:13, SEQ ID NO:19, SEQ ID NO:20, and/or SEQ ID NO:21 listed in Table 2.

In various embodiments, the liposomes encapsulate one or more MG associated antigens, have a negative zeta potential of between −100 mV and 0 mV, and wherein the size of liposomes is between 100 and 1000 nm in diameter. In various embodiments, the liposomes encapsulate one or more MG associated antigens, portions thereof or combinations thereof, wherein the size of the liposomes is between 400 and 800 nm and the liposomes have a negative zeta potential between −30 mV and −80 mV.

In various embodiments, the liposomes encapsulate antigens selected from the group consisting of SEQ ID NOS: 1 to 21 (Table 2), portions or combinations thereof, wherein the size of the liposomes is between 100 and 1000 nm, and the liposomes have a negative zeta potential between-100 mV and 0 mV. In various embodiments, the liposomes are used to induce tolerance in a subject with MG. In various embodiments, the liposome administration is intravenous.

### Antigens

An antigen refers to a discreet portion of a molecule, such as a polypeptide or peptide sequence, a 3-D structural formation of a polypeptide or peptide, a polysaccharide or polynucleotide that can be recognized by a host immune cells. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2).

It is contemplated that the tolerizing therapy described herein is antigen-specific. For example, TIMPs administered as tolerizing therapy encapsulate one or more antigens associated with said tolerizing therapy and associated disease or condition being treated. It is contemplated that the TIMPs used in tolerizing therapy comprise one or more MG antigens, portions thereof, or combinations thereof, and/or MG proteins, portions thereof, or combinations thereof.

In certain embodiments, one, two, three, or a higher number of antigens or antigenic peptides are used in the TIMPs. In certain embodiments, the one or more MG antigens is encapsulated in the TIMP by covalent linkage to the interior surface of the particle (See e.g., US Patent Publication US20190282707, herein incorporated by reference). In certain embodiments, it is contemplated that sequences of two or more MG antigens are linked in a fusion protein and encapsulated within a TIMP described herein. In certain embodiments, it is contemplated that the sequences of two or more MG antigens linked in a fusion protein and encapsulated within a TIMP are selected from table 2. In certain embodiments, it is contemplated that a TIMP encapsulates a fusion protein containing linked antigens and individual antigens. In certain embodiments, the two or more MG associated antigens are linked together by one more linkers that are susceptible to cleavage by specific proteases. Methods for making TIMPs with linked epitopes are described in US Patent Publication US20190365656, herein incorporated by reference. Linking MG-associated antigens improves their solubility and handling for encapsulation within a TIMP.

MG associated proteins (Table 1) include, e.g., Acetylcholine receptor subunit alpha (UNIPROT ID E7ENE5), Acetylcholine receptor subunit alpha (UNIPROT ID P02708), Nicotinic cholinergic receptor alpha 1 (UNIPROT ID Q53SH4), Acetylcholine receptor subunit epsilon (UNIPROT IDQ04844), Acetylcholine receptor subunit delta (UNIPROT ID Q07001), Acetylcholine receptor subunit beta (UNIPROT ID P11230), Acetylcholine receptor subunit gamma (UNIPROT ID P07510), Agrin (UNIPROT ID 000468), Muscle skeletal receptor tyrosine-protein kinase (UNIPROT ID 015146), Cortactin (UNIPROT ID Q14247), Titin (UNIPROT ID Q8WZ42), Ryanodine receptor 1 (UNIPROT ID P21817), Ryanodine receptor 2 (Q92736), Ryanodine receptor 3 (Q15413), Frizzled-9 (UNIPROT ID 000144), Segment polarity protein disheveled homolog DVL-1 (UNIPROT IDO14640), Low-density lipoprotein receptor-related protein 4 (UNIPROT ID 075096), Neuronal acetylcholine receptor subunit beta-2 (UNIPROT ID P17787), Choline O-acetyltransferase (UNIPROT ID P28329), Neuronal acetylcholine receptor subunit alpha-5 (UNIPROT ID P30532), Neuronal acetylcholine receptor subunit beta-4 (UNIPROT ID P30926), Neuronal acetylcholine receptor subunit alpha-3 (UNIPROT ID P32297), Neuronal acetylcholine receptor subunit alpha-7 (UNIPROT ID P36544), Neuronal acetylcholine receptor subunit alpha-4 (UNIPROT ID P43681), Neuronal acetylcholine receptor subunit beta-3 (UNIPROT ID Q05901), Acetylcholine receptor subunit delta (UNIPROT ID Q07001), Serine/threonine-protein kinase (UNIPROT ID Q13153), 43 kDa receptor-associated protein of the synapse (RAPsyn) (UNIPROT ID Q13702), Neuronal acetylcholine receptor subunit alpha-2 (UNIPROT ID Q15822), Neuronal acetylcholine receptor subunit alpha-6 (UNIPROT ID Q15825), Protein Dok-7 (Downstream of tyrosine kinase 7) (UNIPROT ID Q18PE1), Protein RIC-3 (Resistant to inhibitor of cholinesterase 3) (UNIPROT ID Q7Z5B4), Pleckstrin homology-like domain family B member 2 (Protein LL5-beta) (UNIPROT ID Q86SQ0), AchR epsilon subunit (UNIPROT ID Q8N731), Neuronal acetylcholine receptor subunit alpha-10 (Nicotinic acetylcholine receptor subunit alpha-10) (UNIPROT ID Q9GZZ6).

TABLE 1

| Protein name | Gene name | UNIPROT ID |
|---|---|---|
| Acetylcholine receptor subunit alpha | AChRa | E7ENE5 |
| Acetylcholine receptor subunit alpha | AChR alpha subunit | P02708 |
| Nicotinic cholinergic receptor alpha 1 | AChRal | Q53SH4 |
| Acetylcholine receptor subunit epsilon | AChRe | Q04844 |
| Acetylcholine receptor subunit delta | ACHRδ | Q07001 |
| Acetylcholine receptor subunit beta | ACHRβ | P11230 |
| Acetylcholine receptor subunit gamma | ACHRγ | P07510 |
| Agrin | AGRN | O00468 |
| Muscle, skeletal receptor tyrosine-protein | MUSK | O15146 |
| kinase | | |
| Cortactin | CTTN | Q14247 |
| Titin | TTN | Q8WZ42 |
| Ryanodine receptor 1 | RYR1 | P21817 |
| Ryanodine receptor 2 | RYR2 | Q92736 |
| Ryanodine receptor 3 | RYR3 | Q15413 |
| Frizzled-9 (Fz-9) (hFz9) (FzE6) (CD antigen | FZD9 FZD3 | O00144 |
| CD349) | | |
| Segment polarity protein dishevelled homolog | DVL1 | O14640 |
| DVL-1 (Dishevelled-1) (DSH homolog 1) | | |
| Low-density lipoprotein receptor-related protein | LRP4, KIAA0816, | O75096 |
| 4 (LRP-4) (Multiple epidermal growth factor- | LRP10 MEGF7 | |
| like domains 7) | | |
| Neuronal acetylcholine receptor subunit beta-2 | CHRNB2 | P17787 |
| Choline O-acetyltransferase (CHOACTase) | CHAT | P28329 |
| (ChAT) (Choline acetylase) (EC 2.3.1.6) | | |
| Neuronal acetylcholine receptor subunit alpha-5 | CHRNA5 NACHRA5 | P30532 |
| Neuronal acetylcholine receptor subunit beta-4 | CHRNB4 | P30926 |
| Neuronal acetylcholine receptor subunit alpha-3 | CHRNA3 NACHRA3 | P32297 |
| Neuronal acetylcholine receptor subunit alpha-7 | CHRNA7 NACHRA7 | P36544 |
| Neuronal acetylcholine receptor subunit alpha-4 | CHRNA4 NACRA4 | P43681 |
| Neuronal acetylcholine receptor subunit beta-3 | CHRNB3 | Q05901 |
| Acetylcholine receptor subunit delta | CHRND ACHRD | Q07001 |
| Serine/threonine-protein kinase PAK 1 (EC | PAK1 | Q13153 |
| 2.7.11.1) (Alpha-PAK) (p21-activated kinase 1) | | |
| (PAK-1) (p65-PAK) | | |
| 43 kDa receptor-associated protein of the | RAPSN RNF205 | Q13702 |
| synapse (RAPsyn) (43 kDa postsynaptic | | |
| protein) (Acetylcholine receptor-associated 43 | | |
| kDa protein) (RING finger protein 205) | | |
| Neuronal acetylcholine receptor subunit alpha-2 | CHRNA2 | Q15822 |
| Neuronal acetylcholine receptor subunit alpha-6 | CHRNA6 | Q15825 |
| Protein Dok-7 (Downstream of tyrosine kinase | DOK7 C4orf25 | Q18PE1 |
| 7) | | |
| Protein RIC-3 (Resistant to inhibitor of | RIC3 | Q7Z5B4 |
| cholinesterase 3) | UNQ720/PRO1385 | |
| Pleckstrin homology-like domain family B | PHLDB2 LL5B | Q86SQ0 |
| member 2 (Protein LL5-beta) | | |
| AchR epsilon subunit | CHRNE | Q8N731 |
| Neuronal acetylcholine receptor subunit alpha- | CHRNA10 | Q9GZZ6 |
| 10 (Nicotinic acetylcholine receptor subunit | NACHRA10 | |
| alpha-10) (NACHR alpha-10) | | |

MG associated antigens include AChR antigens with amino acid sequences selected from Table 2. In various embodiments, TIMP-MG encapsulates antigens comprising a combination of SEQ ID NO:3, SEQ ID NO:5, SEQ ID NO:8, SEQ ID NO:13, SEQ ID NO 19, SEQ ID NO:20, SEQ ID NO:21 set out in Table 2.

TABLE 2

| SEQ ID | | |
|---|---|---|
| NO: | Name | Sequence |
| SEQ ID 1 | AChRα (48-67) | QIVTTNVRLKQQWVDYNLKW |
| SEQ ID 2 | AChRα (101-120) | AIVKFTKVLLQYTGHITWTP |
| SEQ ID 3 | AChRα isoform α | STHVMPNWVRKVFIDTIPNIMFFSTMKRPSR |
| | (324-357) | EKQ |
| SEQ ID 4 | AChRα (419-437) | IIGTLAVFAGRLIELNQQG |
| SEQ ID 5 | AChRϵ (116-130) | IDGQFGVAYDANVLV |
| SEQ ID 6 | AChRϵ (201-215) | ENGEWAIDFCPGVIR |
| SEQ ID 7 | AChRϵ (236-250) | IRRKPLFYVINIIVP |
| SEQ ID 8 | AChRϵ (414-435) | GAAAPEVRCCVDAVNFVAESTR |
| SEQ ID 9 | AChRα (125-163) | KSYCEIIVTHFPFDEQNCSMKLGTWTYDGS |
| | | VVAINPESD |
| SEQ ID 10 | AChRα (386-411) | MKSDQESNNAAAEWKYVAMVMDHILL |
| SEQ ID 11 | AChRα (256-269) | FLLVIVELIPSTSS |
| SEQ ID 12 | AChRα (167-183) | AIFKSYCEIIVTHFPFD |
| SEQ ID 13 | AChRα (43-58) | EDHRQVVEVTVGLQLI |
| SEQ ID 14 | AChRα (123-138) | IHIPSEKIWRPDLVLY |
| SEQ ID 15 | AChRα (112-127) | WNPDDYGGVKKIHIPS |
| SEQ ID 16 | AChRα (76-91) | LKQQWVDYNLKWNPDD |
| SEQ ID 17 | AChRα (66-76) | YNLKWNPDDYGGVKKIHI |
| SEQ ID 18 | AChRα (351-368) | IEGIKYIAETMKSDQESN |
| SEQ ID 19 | AChRα (68-113) | QIVTTNVRLKQQWVDYNLKWNPDDYGGV |
| | | KKIHIPSEKIWRPDLVLY |
| SEQ ID 20 | AChRα (121-158) | AIVKFTKVLLQYTGHITWTPPAIFKSYCEIIV |
| | | THFPFD |
| SEQ ID 21 | AChRϵ (201-250) | ENGEWAIDFCPGVIRRHHGGATDGPGETDV |
| | | IYSLIIRRKPLFYVINIIVP |

Emulsions occur in many forms of processing and are used extensively by the food, cosmetics and drug delivery. Oil-water (single), water-oil-water (double) emulsion are methods by which PLGA can be used to encapsulate hydrophobic and hydrophilic drugs in micro- or nanoscale form. In summary, PLGA is dissolved into an organic phase (oil) that is emulsified with a surfactant or stabilizer (water). Hydrophobic drugs and/or other agents are added directly to the oil phase, whereas hydrophilic drugs and/or other agents (water) may be first emulsified with the polymer solution prior to formation of particles. High intensity homogenization (e.g., sonication bursts) facilitate the formation of small polymer droplets. The resulting emulsion is added to a larger aqueous phase and stirred for several hours, which allows the solvent to evaporate. Hardened nanoparticles are collected and washed by centrifugation. In certain embodiments, hardened emulsion particles can be obtained through evaporation of the oil phase.

“Water-in-oil-in-water” (W/O/W) emulsion is an example of a double emulsion, in which dispersions of small water droplets within larger oil droplets are themselves dispersed in a continuous aqueous phase. Because of their compartmentalized internal structure, double emulsions can provide advantages over simple oil-in-water emulsions for encapsulation, such as the ability to carry both polar and non-polar cargos (pharmaceutical/biological agent, e.g., proteins), and improved control over release of therapeutic molecules. The preparation of double emulsions typically requires surfactants or their mixtures for stability. The surfactants stabilize droplets subjected to extreme flow, leading to direct, mass production of robust double nanoemulsions that are amenable to nanostructured encapsulation applications in various industries. In one example, a double emulsion process involves generating a primary emulsion by mixing an aqueous solution of a pharmaceutical/biological agent(s) with a solution including a polymer resulting in a water-in-oil primary emulsion. The primary emulsion is then mixed with a solution including one or more surfactants to form an oil-in-water secondary emulsion. The secondary emulsion is then hardened by evaporation to remove the solvent(s) resulting in hardened polymeric nanoparticles encapsulating the pharmaceutical/biological agent(s).

“Homogenization” as used herein relates to an operation using a class of processing equipment referred to as homogenizers that are geared towards reducing the size of droplets in liquid-liquid dispersions. Factors that affect the particle or droplet size include but are not limited to the type of emulsifier, emulsifier concentration, solution conditions, and mechanical device (homogenizing power; pressure, rotation speed, time). Non-limiting examples of homogenizers include high speed blender, high pressure homogenizers, colloid mill, high shear dispersers, ultrasonic disruptor membrane homogenizers, and ultrasonicators. Mechanical homogenizers, manual homogenizers, sonicators, mixer mills, vortexers, and the like may be utilized for mechanical and physical disruption within the scope of the disclosure.

“Batch size” as used herein relates to the scale of manufacture depending on the weight of the particles in the final product. The manufacturing process can be altered, scaled up or scaled down. The manufacturing process can be altered, scaled up or scaled down by altering the amount or volume of the solvent, antigens/proteins, polymer, surfactants, stabilizers, cryoprotectants or excipients. The manufacturing process can be scaled up or down by altering the time of homogenization, sonication, evaporation, filtration, concentration, washing or lyophilization. Number of vials filled with TIMP-MG will vary depending on batch size. About 1, 5, 50, 500, 5000, 50,000, or 500,000 vials are filled including all values lying within this range. Amount of TIMP-MG filled in each vial is about 0.01 mg, about 0.1 mg, about 1 mg, about 10 mg, about 100 mg, about 200 mg, or about 250 mg including all values lying within this range.

Methods for determining protein content in the particles or in solution include ELISA, Mass Spectrometry, HPLC, CBQCA, and Western Blot.

Molecular Probes CBQCA Protein Quantitation Kit provides a rapid and highly sensitive method for the quantitation of proteins in solution. The kit utilizes the ATTO-TAG CBQCA reagent (3-(4-carboxybenzoyl) quinoline-2-carboxaldehyde) originally developed as a chromatographic derivatization reagent for amines. This reagent has also proven extremely useful for quantitating amines in solution, including the accessible amines in proteins. The ATTO-TAG CBQCA reagent is virtually non-fluorescent in aqueous solution; however, in the presence of cyanide, it reacts with primary amines such as those found in proteins to form highly fluorescent derivatives.

### Acid

An acid is a molecule or ion capable of either donating a proton (i.e. hydrogen ion, H⁺), or forming a covalent bond with an electron pair.

An aqueous solution of an acid has a pH less than 7. A lower pH means a higher acidity, and thus a higher concentration of positive hydrogen ions in the solution. Chemicals or substances having the property of an acid are said to be acidic.

In various embodiments the acid is acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid.

### Base

A base is a chemical species that donates electrons, accepts protons, or releases hydroxide (OH−) ions in aqueous solution.

An aqueous solution of a base has a pH more than 7.0 and up to 14.0. A higher pH means a higher basicity, and thus a higher concentration of negative hydroxide ions in the solution. Chemicals or substances having the property of a base are said to be basic.

In various embodiments the base is barium hydroxide, calcium hydroxide, chromium hydroxide, potassium hydroxide, ammonium hydroxide, zinc hydroxide, barium hydroxide, sodium bicarbonate, methylamine, diethylamine, sodium hydroxide, magnesium hydroxide, ammonium bicarbonate, ammonia aluminium hydroxide, sodium carbonate, magnesium hydroxide, zinc hydroxide, ferrous hydroxide, acetone, lithium hydroxide, pyridine, rubidium hydroxide.

### Methods of Use

Provided herein is a method of treating MG in a subject comprising administering to the subject TIMP-MG, wherein TIMP-MG is administered at a dose level determined based on the subject's weight. It is also contemplated that TIMP-MG may be administered at a fixed dosage irrespective of the subject's weight. In various embodiments, contemplated is a method of treating MG in a subject comprising administering to the subject TIMP-MG, wherein TIMP-MG is administered at a dose of 0.001 to 12 mg/kg based on the subject's weight or at a fixed dose between 0.1 mg and 800 mg. Also provided herein is a method of reducing an inflammatory immune response to MG antigens in a subject suffering from MG comprising administering to the subject TIMP-MG, wherein TIMP-MG is administered at a dose of 0.001 mg/kg to 12 mg/kg based on the subject's weight or at a fixed dose between 0.1 mg and 800 mg.

Also contemplated, the TIMP-MG is administered at a dose from about 0.001 to about 10 mg/kg, from about 0.005 to about 12 mg/kg, from about 0.01 to about 12 mg/kg, from about 0.05 to about 12 mg/kg, from about 0.1 to about 12 mg/kg, from about 0.5 to about 10 mg/kg, from about 1 mg/kg to about 8 mg/kg, from about 1.5 to about 10 mg/kg, from about 2 mg/kg to about 12 mg/kg, from about 2 mg/kg to about 10 mg/kg, from about 3 mg/kg to about 10 mg/kg, from about 4 to about 10 mg/kg, from about 4 to about 12 mg/kg, or from about 5 to about 12 mg/kg. Optionally, the TIMP-MG is administered in a dose of about 0.001 mg/kg, about 0.0025 mg/kg, about 0.005 mg/kg, about 0.01 mg/kg, about 0.025 mg/kg, about 0.05 mg/kg, about 0.1 mg/kg, about 0.25 mg/kg, about 0.5 mg/kg, about 1.0 mg/kg, about 2.0 mg/kg, about 3.0 mg/kg, about 4.0 mg/kg, about 5 mg/kg, about 6 mg/kg, about 8.0 mg/kg, about 10 mg/kg, or about 12 mg/kg. Alternatively, TIMP-MG is administered at a fixed dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In another embodiment, TIMP-MG is administered at a concentration of between about 0.0005 mg/mL and about 50 mg/mL, optionally about 0.0005 mg/mL, 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 3.25 mg/mL, 3.5 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL. In various embodiments, TIMP-MG is administered via intravenous infusion lasting about 1, 2, 3, 4, 5, 6, 7, or 8 hours.

It is contemplated that the TIMP-MG is administered in a single dose or in multiple doses. In various embodiments, TIMP-MG is administered once weekly, once every two weeks, once every three weeks, once every 4 weeks, once every two months, once every three months, once every 4 months, once every 5 months, once every 6 months, once per year, once every two years, once every three years, once every four years, once every five years, once every six years, once every seven years, once every eight years, once every nine years, or once every ten years. In certain embodiments, TIMP-MG is administered in two doses one-week apart.

In various embodiments, a booster dose of TIMP-MG is administered in a single dose or in multiple doses following the original TIMP-MG administration. In various embodiments, the booster dose is administered once weekly, once every two weeks, once every three weeks, once every 4 weeks, once every two months, once every three months, once every 4 months, once every 5 months, once every 6 months, or once per year. In various embodiments, TIMP-MG is administered in two doses one-week apart followed by a booster dose administered as a single dose once every three months. The booster dose is administered when needed as determined by an analytical test or screening methods used to test weakening of immunological tolerance. Methods of tracking maintenance or weakening of immunological tolerance are described below and/or in patent publication WO2022221622 herein by reference.

In various embodiments, TIMP-MG is administered intravenously, subcutaneously, intramuscularly, intraperitoneally, intranasally, or orally. It is contemplated that if TIMP-MG is given intravenously, it can be via intravenous infusion lasting about 1, 2, 3, 4, 5, 6, 7, 8, 12, 18 or 20 or.

In various embodiments, TIMP-MG administration is based on one or more of the following: subject's profile/HLA haplotype, Myasthenia Gravis Foundation of America (MGFA) clinical classification score (Class I, Class II, Class III, Class IV, Class V), baseline MG-ADL score (e.g. between 1 and 24 including all values lying within this range), baseline QMG score (e.g. between 1 and 39 including all values lying within this range), baseline MGC score (e.g between 1 and 50 including all values lying within this range), symptoms (e.g. ocular or non-ocular symptoms), concurrent or previously administered medications/therapeutics/drugs, concurrent or active diseases, presence/levels of anti-AChR antibodies, presence/levels of anti-MuSK antibodies or other MG associated antibodies. In various embodiments, the HLA haplotype is selected from HLA-DRB1*03, HLA-DRB3*01, or HLA-DQB1*02. In some embodiments, the MGFA clinical classification score is III-IV or II-IV). In various embodiments, the MG-ADL score is ≥6 with ≥50% of the score based on non-ocular symptoms). In various embodiments, the QMG score is ≥11.

In various embodiments, TIMP-MG is infused at escalating rates. In various embodiments, the escalating rate doubles after 15 minutes of initial infusion. In various embodiments, the escalating rate doubles after the second 15 minutes of initial infusion e.g., is 4 times the initial infusion rate. In some embodiments, the TIMP-GLIA is infused over approximately 2.5 hours at approximately 20 mL/hour for the first 15 minutes, 40 mL/hour for the next 15 minutes, and 80 mL/hour for the duration of the infusion.

In various embodiments, the disclosure provides a method of treating MG or MG related symptoms, in a subject comprising administering to the subject a composition comprising TIMP-MG alone or in combination with a therapeutic. In various embodiments, the therapeutic is a cholinesterase inhibitor, steroid, corticosteroid, nonsteroidal immunosuppressive agent, immunomodulatory agent, therapeutic plasma exchange (plasmapheresis), intravenous immunoglobulin (IVIG), chloride ion channel inhibitor, monoclonal antibody, proteasome inhibitors, cytokine and chemokine targeting therapy, microRNA inhibitor, siRNA, JAK inhibitor, BTK inhibitor, complement inhibitor, IgG degrader, neonatal Fc receptor (FcRn) inhibitor, chimeric antigen receptor (CAR) T-cell therapy, regulatory T-cell (Treg) therapy, hematopoietic stem cell transplant, B-cell targeting therapy, or surgical treatment. In various embodiments, the therapies are selected from the group comprising azathioprine, sirolimus, edrophonium, neostigmine, pyridostigmine, cyclosporine, mycophenolate mofetil, calcineurin inhibitor, levamisole, chlorambucil, cyclophosphamide, tacrolimus, methotrexate, eculizumab, ravulizumab, efgartigimod alfa, rozanolixizumab, zilucoplan, CD20 targeting therapy, CD19 targeting therapy, CD40/CD40L targeting therapy, B cell targeting factor (BAFF) targeting therapy, B cell maturation antigen (BCMA) targeting therapy, anti-IL6 therapy, anti-IFN therapy, anti-thymocyte globulin, factor D inhibitor, amifampridine, batoclimab, inebilizumab, nipocalimab, pozelimab, rituximab, satralizumab, tocilizumab, tofacitinib, tolebrutininb. In various embodiments the steroid or corticosteroid, is selected from the group comprising beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, oxymetazoline hydrochloride, or hydrocortisone. In various embodiments, the surgical treatment is a thymectomy. In various embodiments the therapeutic is administered prior to, concomitantly with or after the administration of TIMP-MG. In various embodiments, the therapeutic is administered 1, 2, 3, 4, 5, 6, or 7 days prior to administration of TIMP-MG. In various embodiments, the therapeutic is administered 1, 2, 3, or 4 weeks prior to administration of TIMP-MG. In various embodiments, the therapeutic is administered 1, 2, 3, 4, 5, 6, 7, 8, 9, 10, 11, or 12 prior to administration of TIMP-MG. In various embodiments, the therapeutic is administered 1, 2, 3, 4, 5, 6, 7, 8, 9, 10, 11, or 12 years prior to administration of TIMP-MG. In various embodiments, the therapeutic is administered 1, 2, 3, 4, 5, 6, or 7 days subsequent to administration of TIMP-MG. In various embodiments, the therapeutic is administered 1, 2, 3, or 4 weeks subsequent to administration of TIMP-MG. In various embodiments, the therapeutic is administered 1, 2, 3, 4, 5, 6, 7, 8, 9, 10, 11, or 12 months subsequent to administration of TIMP-MG. In various embodiments, the therapeutic is administered 1, 2, 3, 4, 5, 6, 7, 8, 9, 10, 11, or 12 years subsequent to administration of TIMP-MG.

TIMP-MG therapy is contemplated to relieve, lessen or ameliorate one or more symptoms of MG. Symptoms of MG include, but are not limited to droopy eyelids (ptosis), double vision (diplopia), difficulty making facial expressions, problems chewing, difficulty swallowing, slurred speech (dysarthria), shortness of breath, breathing difficulties, muscle fatigue, skeletal muscle weakness, neck weakness, limb weakness, dysphagia, dysphonia, weakened neuromuscular junctions, impaired synaptic signal transduction, muscle damage, rescue therapy with IVIG/PLEX, complement activation, anti-Musk antibodies, anti-AChR antibodies, increased activated CD4+ cells in PBMCs, and increased activated CD8+ T-cells in PBMCs, increased Myasthenia Gravis Activities of Daily Living Score, increased Quantitative MG Score, MG Quality of Life 15-revised score, and MG Composite Score.

TIMP-MG therapy is also contemplated to reduce, shorten or ameliorate the duration and severity of an inflammatory immune response to one or more MG antigens in a subject. An inflammatory immune response includes a T cell response, B cell response, Th1 response, myeloid cell response, and/or an antibody response. In various embodiments, the efficacy of TIMP-MG at relieving one or more symptoms of MG and/or reducing the duration and severity of an inflammatory immune response to one or more MG antigens is determined from the assay of one or more biological samples from the subject as described herein.

Provided herein is a method of inducing tolerance to a subject in need thereof comprising administering in a subject a composition comprising negatively charged particles encapsulating an antigen, wherein the antigen is one or more MG associated antigens, portions, or combinations thereof.

In various embodiments, the subject is a subject who has MG, a subject who is receiving therapy for MG, or a subject who will receive therapy for MG.

In various embodiments, the TIMP-MG particles comprise one or more MG associated antigens, portions thereof, combinations thereof, or one or more antigenic epitopes thereof. In various embodiments, the TIMP-MG particles comprise one or more MG associated proteins, portions thereof, combinations thereof, or one or more antigenic epitopes thereof.

If TIMP-MG particles comprising a single antigen are administered in combination with another carrier particle comprising a different antigen, or second agent, the particles and or second agent can be administered concurrently or sequentially. Concomitant or concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks. It is further contemplated that the therapeutics are administered in a separate formulation and administered concurrently or concomitantly, with concurrently referring to agents given within 30 minutes of each other. Prior administration refers to administration of a therapeutic within the range of one week prior to treatment with a TIMP-MG particle, up to 30 minutes before administration of a TIMP-MG particle. Subsequent administration is meant to describe administration from 30 minutes after treatment up to one week after administration.

### Screening Methods

It is contemplated that induction of and maintenance of immunological tolerance is monitored in a subject suffering from MG, wherein the subject is treated, or about to undergo treatment, with antigen-specific tolerizing therapy comprising TIMP-MG particles as described herein.

Methods of screening for cell types, cytokines or other measures of tolerance from a subject undergoing tolerizing therapy as described herein are known in the art. Methods of assessing tolerance are done using such techniques as flow cytometry, radioimmunoassay, Mass Cytometry (CyTOF), ELISA, ELISPOT, in vitro/ex vivo cell stimulation assays (including, but not limited to, cell proliferation assays, macrophage stimulation assays), measuring autoantibodies or measuring Ig serotype, e.g., by ImmunoCap assay.

In various embodiments, the immune tolerance status of a subject is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of cell-surface proteins, extracellular proteins, intracellular proteins, nucleic acids, metabolites, enzymes, and/or combinations thereof relevant in the disease or disorder.

Cells assayed from the biological sample include immune cells, non-immune cells, and/or combinations thereof. Immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells. In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

One aspect of a subject's immune tolerance status, and immune signature, is determined by analyzing one or more proteins from one or more biological sample(s) from the subject. In various embodiments, the proteins are cytokines and/or chemokines. In various embodiments the proteins are cell signaling proteins. In various embodiments, the cytokines and chemokines are selected from the group consisting of IL-1a, IL-1B, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1a, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, soluble CD14, and/or combinations thereof. In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, and EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. In various embodiments the immunoglobulins are MG antigen specific. In various embodiments the immunoglobulins are AChR antigen specific. In various embodiments the immunoglobulins are Musk antigen specific. Several methods for the detection of immunoglobulins from a biological sample have been described in the literature including radioimmunoassay, ELISA and ImmunoCap.

One aspect of a subject's immune tolerance status, and immune signature, is determined by analyzing one or more metabolites from the biological sample(s). In various embodiments, the metabolite is an inflammatory metabolite. In various embodiments, the metabolite is an anti-inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), lipoxin A4, and lipoxin B4. In various embodiments, examples of anti-inflammatory metabolites include kynurenine, 3-hydroxy kynurenine, 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine.

A list of human metabolites that can be assayed from a biological sample can be found in the literature including in (Psychogios et al., 2011), (Wishart et al., HMDB: the Human Metabolome Database. Nucleic Acids Res. 2007 January; 35 (Database issue): D521-6, 2007), and the Human Metabalome Database (HMDB) and are incorporated herein by reference.

One aspect of a subject's immune tolerance status, and immune signature, is determined by analyzing one or more cell-surface proteins from a biological sample(s). In various embodiments, the cell-surface proteins include CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2Rβ, IL-4R, IL-5Rα, CSF2Rβ, IL-6Rα, gp130, IL-7Rα, IL-9R, IL-10R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22RB, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TLIA, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULTI, RAE1 α,β, γ, δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In certain embodiments, the subject's tolerance status is determined by analyzing nucleic acids from the biological sample(s). In various embodiments, the nucleic acids are DNA and/or RNA, including, but not limited to, single stranded DNA, double stranded DNA, mRNA, rRNA, tRNA, siRNA, miRNA, long non-coding RNAs (long ncRNAs, lncRNA), and non-coding RNA (ncRNA), mitochondrial RNA. In various embodiments, the miRNA is selected from the group comprising miR-146, miR-15a, miR-125a-5p, miR-15-5p, miR-21-5p, miR-139-5p, miR-452-5p, miR-7-5p, miR-548k, miR-653, miR-146, miR612, miR-3651, miR-3653, miR-15b, miR-15b, miR-145, miR-20b, miR-150-5p, miR-21-5p, miR-20b, miR-30e-5p, miR-145, miR-146, miR-181c, miR320a, miR155, miR-181a, miR-21-5p, miR-27a-3p or miR-30e-5p. In certain embodiments, treatment with TIMP-MG decreases the levels of miRNAs compared to baseline. In certain embodiments, treatment with TIMP-MG increases the levels of miRNAs compared to baseline. In certain embodiments, treatment with TIMP-MG decreases miRNA levels by 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement. In certain embodiments, treatment with TIMP-MG increases miRNA levels by 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement.

In various embodiments, the subject's immune tolerance status is determined by assaying gene expression from the biological sample(s). In various embodiments, the immune tolerance status is determined by assaying gene expression associated with immune function, an antibody, foreign body response, metabolism, apoptosis, cell death, necrosis, ferroptosis, autophagy, cell migration, endocytosis, phagocytosis, pinocytosis, tight-junction regulation, cell adhesion, differentiation, and/or combinations thereof. In various embodiments, the immune tolerance status is determined by assaying gene expression associated with immune suppression. In various embodiments, the immune tolerance status is determined by assaying gene expression associated with immune activation. In various embodiments, the immune tolerance status is determined by assaying gene expression associated with immune regulatory functions. In various embodiments, nucleic acid analysis is used to generate an immune tolerance signature. Several methodologies have been described in the literature for high-throughput gene expression analysis including RNA sequencing (RNA-seq), single-cell RNA sequencing (scRNA-seq), exome sequencing, and microarray-based analyses.

The biological sample is optionally assayed after in vivo and/or ex vivo stimulation with one or more stimuli such as an antigen, an allergen, and one or more activating agents. It is contemplated that the T cells, B cells, and immunoglobulins used in the assay are antigen specific. Exemplary T cells include effector memory T cells, antigen specific T cells, activated antigen specific T cells, Th1 cells, pathogenic Th2a+ cells, Th17 cells, T follicular helper (TFH) cells, TH0 cells, proportion of antigen specific CD4+ T cells (disease specific T cell/effector memory T cell), proportion of activated antigen specific CD4+ T cells (activated disease specific T cell/disease specific T cell), proportion of antigen specific CD8+ T cells (CD154-CD137+CD8+ T cells/total CD8+ T cells), proportion of activated antigen specific CD8+ T cells (CD38+CD154-CD137+CD8+ T cells/CD154-CD137+CD8+ T cells), proportion of antigen specific T regulatory cells (CD154-CD137+CD127-CD25+TIGIT+ effector memory CD4+ T cells/CD154-CD137+ effector memory CD4+ T cells), or other antigen-specific T cells. B cells include effector B cells, memory B cells, plasma cells, and regulatory B (Breg) cells. In certain embodiments, T cells are identified based on the expression of proteins described in Table 3.

TABLE 3

| Cell Type | Expression Marker |
|---|---|
| Effector memory (EM) T cell | CD45RA^{LO} CD4⁺ |
| Disease specific T cell | CD45RA^{LO} CD4⁺ CD154⁺ CD137⁺ |
| Activated disease specific T cell | CD45RA^{LO} CD4⁺ CD154⁺ CD137⁺ CD38⁺ |
| Th2 cell | CD45RA^{LO} CD4⁺ CD154⁺ CD137⁺ CXCR4 |
| Th1 cell | CXCR5⁻ CRTH2⁻ CXCR3⁺ CCR6⁻ |
| Th17 cell | CXCR5⁻ CRTH2⁻ CXCR3⁻ CCR6⁺ |
| TFH cell | CD45RA^{LO} CD4⁺ CXCR5⁺ |
| Treg | CD154⁻ CD127⁻ CD25⁺ TIGIT⁺ CD137⁺ GARP^{+/-} |
| | OX40^{+/-} |
| Th0 cell | CXCR5⁻ CRTH2⁻ CXCR3⁻ CCR6⁻ CCR4⁻ |
| Disease specific CD8 T cells | CD8⁺ CD137⁺ CD69⁺ CD355⁺ |
| Activated antigen specific CD8 T cell | CD8⁺ CD137⁺ CD69⁺ CD355⁺ CD38⁺ |
| Exhausted T cells | CD4⁺ TIGIT⁺ KLRG1⁺ PD-1⁺ |

The immune tolerance signature of a subject is generated using one or more of the following parameters assayed from one or more biological samples obtained from the subject and stimulated in vivo and/or ex vivo:
A. proportion of effector T cells in the total T cell population,
B. proportion of Treg cells in the total T cell population,
C. proportion of effector B cells in the total B cell population,
D. levels of specific IgG, and/or IgM,
E. levels of inflammatory cytokines and chemokines,
F. levels of anti-inflammatory cytokines and chemokines,
G. levels of liver enzymes,
H. levels of inflammatory metabolites, and
I. levels of anti-inflammatory metabolites.

The immune tolerance signature is indicative of maintenance of immune tolerance if 1, 2, 3, 4, 5, 6, 7, 8, or 9 parameters listed in (a)-(i) above indicate maintenance of immune tolerance. In various embodiments, the immune tolerance signature is indicative of maintenance of immune tolerance if at least 2/9 parameters listed in (a)-(i) indicate maintenance of immune tolerance. In various embodiments, the subject is determined to not require treatment with TIMPs if 1, 2, 3, 4, 5, 6, 7, 8, or 9 parameters listed in (a)-(i) above indicate maintenance of immune tolerance. In various embodiments, the subject is determined to not require treatment with TIMPs if at least 3/9 parameters listed in (a)-(i) above indicate maintenance of immune tolerance.

The immune tolerance signature of a subject generated using one or more parameters described herein indicates weakening and/or absence of immune tolerance prior to or after treatment with TIMPs, if:
a. the proportion of effector T cells in the total T cell population is between 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), relative to the subject's baseline measurement and/or relative to a healthy subject and/or
b. the proportion of Treg cells in the total T cell population is between 0.01-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values) relative to the subject's baseline measurement and/or relative to a healthy subject, and/or
c. the proportion of effector B cells in the total B cell population is between 0.01%-100% (e.g about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), relative to the subject's baseline measurement and/or relative to a healthy subject and/or
d. the levels of IgG and/or IgM are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
e. levels of inflammatory cytokines/chemokines are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
f. levels of anti-inflammatory cytokines and chemokines are decreased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
g. levels of liver enzymes are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
h. levels of inflammatory metabolites are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
i. levels of anti-inflammatory metabolites are decreased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

The efficacy of TIMP-MG at relieving one or more symptoms of MG and/or reducing the duration and severity of an inflammatory immune response to MG antigens is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, enzymes, and/or combinations thereof.

In various embodiments, the efficacy of TIMP-MG at relieving one or more symptoms of MG and/or reducing the duration and severity of an inflammatory immune response to MG antigens is determined based on the assay of cells from one or more biological samples from the subject pre- and post-treatment with TIMP-MG. In various embodiments, the cells are immune cells, non-immune cells, and/or combinations thereof. In various embodiments, immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

In various embodiments, the efficacy of TIMP-MG at relieving one or more symptoms of MG and/or reducing the duration and severity of an inflammatory immune response to MG antigens is determined based on the assay of cell surface proteins from one or more biological samples from the subject pre- and post-treatment with TIMP-MG. In various embodiments, the cell surface proteins are selected from the group consisting CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2Rβ, IL-4R, IL-5Rα, CSF2Rβ, IL-6Rα, gp130, IL-7Rα, IL-9R, IL-10R, IL-12Rβ1, IL-12Rβ2, IL-13Ra1, IL-13Rα2, IL-15Rα, IL-21R, IL23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP,α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULTI, RAE1 α,β, γ, δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In various embodiments, treatment with TIMP-MG decreases the expression of inflammatory cell surface proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject. In various embodiments, treatment with TIMP-MG increases the expression of anti-inflammatory cell surface proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG at relieving one or more symptoms of MG and/or reducing the duration and severity of an inflammatory immune response to MG antigens is determined based on the assay of proteins from one or more biological samples from the subject pre- and post-treatment with TIMP-MG. In various embodiments, the proteins are cytokines and/or chemokines. In various embodiments the proteins are cell signaling proteins. In various embodiments, the cytokines and chemokines are selected from the group consisting of IL-1a, IL-1B, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, and/or combinations thereof.

In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, caspase 1, caspase 2, caspase 3, caspase 4, caspase 5, caspase 6, caspase 7, caspase 8, Caspase 9, caspase 10, caspase 11, caspase 12, caspase 13, caspase 14, BCL-2, BCL-XL, MCL-1, CED-9, A1, BFL1, BAX, BAK, DIVA, BCL-XS, BIK, BIM, BAD, BID, and EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. Several methods for the detection of immunoglobulins from a biological sample have been described in the literature including radioimmusonassay, ELISA and ImmunoCap. In various embodiments, treatment with TIMP-MG decreases the levels of inflammatory proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject. In various embodiments, treatment with TIMP-MG increases the levels of anti-inflammatory proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG at relieving one or more symptoms of MG and/or reducing the duration and severity of an inflammatory immune response to MG antigens is determined based on the assay of metabolites from one or more biological samples from the subject pre- and post-treatment with TIMP-MG. In various embodiments, the metabolite is an inflammatory metabolite. In various embodiments, the metabolite is an anti-inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), lipoxin A4, and lipoxin B4. In various embodiments, examples of anti-inflammatory metabolites include kynurenine, 3-hydroxy kynurenine, 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine. In various embodiments, treatment with TIMP-MG decreases the levels of inflammatory metabolites by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject. In various embodiments, treatment with TIMP-MG increases the levels of anti-inflammatory metabolites by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG is determined based on the following assessments pre- and post-TIMP-MG administration:
A. Proportion of antigen specific CD4+ and CD8+ T cells
B. Proportion of activated antigen specific CD4+ and CD8+ T cells
C. Proportion of T regulatory (Treg) cells
D. Proportion of anti-AChR antibodies in serum.
E. Proportion of anti-MuSK antibodies in serum.
F. Myasthenia Gravis Composite (MGC) score
G. Quantitative score of muscle weakness (QMG) Score.
H. MG Activities of Daily Living (MG-ADL) Score.
I. MG Quality of Life 15-revised (MG-QOL15r) Score.
J. Health care utilization by hospital visits (MG exacerbation rate requiring rescue therapy with IVIG/PLEX)
K. Use of alternative MG therapies

In various embodiments, the efficacy of TIMP-MG at improving one or more symptoms of MG is determined from the result of the assay of 1, 2, 3, 4, 5, 6, 7, 8, 9, 10 or 11 parameters listed in (A)-(K) above.

In various embodiments, the efficacy of TIMP-MG is determined based on the assay of antigen specific CD4+ and/or CD8+ T cells. In various embodiments, treatment with TIMP-MG decreases the levels of antigen specific CD4+ and/or CD8+ T cells by 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG is determined based on the assay of activated antigen specific CD4+ and/or CD8+ T cells. In various embodiments, treatment with TIMP-MG decreases the levels of activated antigen specific CD4+ and/or CD8+ T cells by 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG is determined based on the assay of regulatory T cells or antigen specific regulatory T-cells. In various embodiments, treatment with TIMP-MG increases the levels of regulatory T cells or antigen specific regulatory T-cells specific by 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG is determined based on the assay of anti-AChR antibodies. In various embodiments, treatment with TIMP-MG decreases the levels of anti-AChR antibodies by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG is determined based on the assay of anti-MuSK antibodies. In various embodiments, treatment with TIMP-MG decreases the levels of anti-MuSK antibodies by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-MG is determined based on the subject's score on a clinical disease scale. In various embodiments, the clinical disease scale is the QMG Score, Myasthenia Muscle Score (MMS), Besta Neurologic Institute rating scale for MG (INCB-MG), MG Activities of Daily Life (MG-ADL), Manual Muscle Test (MMT), MG Composite (MGC), Oculobulbular Facial Respiratory Score (OBFR), MG Impairment Index (MGII), MG-QOL-15r Score. In various embodiments, TIMP-MG decreases the QMG Score, Myasthenia Muscle Score (MMS), Besta Neurologic Institute rating scale for MG (INCB-MG), MG Activities of Daily Life (MG-ADL), Manual Muscle Test (MMT), MG Composite (MGC), Oculobulbular Facial Respiratory Score (OBFR), MG Impairment Index (MGII), MG-QOL-15r Score. A number of clinical disease scoring systems have been described in the literature and are incorporated herein by reference⁷.

In various embodiments, the efficacy of TIMP-MG is determined based on health care utilization by hospital visits (MG exacerbation rate requiring rescue therapy with IVIG/PLEX). In various embodiments, treatment with TIMP-MG decreases health care utilization by hospital visits, decreases MG exacerbation rate, or rescue therapy with IVIG/PLEX. In various embodiments, TIMP-MG decreases health care utilization by hospital visits, decreases MG exacerbation rate, or rescue therapy with IVIG/PLEX 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, efficacy of TIMP-MG is determined based on the use of alternative MG therapies. In various embodiments, treatment with TIMP-MG decreases use of alternative MG therapies. In various embodiments, the alternative MG therapy is a cholinesterase inhibitor, steroid, corticosteroid, nonsteroidal immunosuppressive agent, immunomodulatory agent, therapeutic plasma exchange (plasmapheresis), intravenous immunoglobulin (IVIG), monoclonal antibody, proteasome inhibitos, cytokine and chemokine targeting therapy, microRNA inhibitor, siRNA, JAK inhibitor, BTK inhibitor, complement inhibitor, IgG degrader, chimeric antigen receptor (CAR) T-cell therapy, regulatory T-cell (Treg) therapy, hematopoietic stem cell transplant, B-cell targeting therapy, or surgical treatment. In various embodiments, the therapies are selected from the group comprising azathioprine, sirolimus, edrophonium, neostigmine, pyridostigmine, cyclosporine, mycophenolate mofetil, calcineurin inhibitor, levamisole, chlorambucil, cyclophosphamide, tacrolimus, methotrexate, eculizumab, ravulizumab, cfgartigimod alfa, rozanolixizumab, zilucoplan, CD20 targeting therapy, CD19 targeting therapy, CD40/CD40L targeting therapy, B cell targeting factor (BAFF) targeting therapy, B cell maturation antigen (BCMA) targeting therapy, anti-IL6 therapy, anti-IFN therapy, anti-thymocyte globulin, factor D inhibitor, amifampridine, batoclimab, inebilizumab, nipocalimab, pozelimab, rituximab, satralizumab, tocilizumab, tofacitinib, tolebrutininb. In various embodiments the steroid or corticosteroid, is selected from the group comprising beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, oxymetazoline hydrochloride or hydrocortisone. In various embodiments, administration of TIMP-MG decreases use of alternative MG therapies 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% relative to the subject's baseline measurement and/or relative to a healthy subject. In various embodiments, the use of alternative MG therapies is reduced or eliminated 1, 2, 3, 4, 5, 6, or 7 days after administration of TIMP-MG. In various embodiments, the use of alternative MG therapies is reduced or eliminated 1, 2, 3, or 4 weeks after administration of TIMP-MG. In various embodiments, the use of alternative MG therapies is reduced or eliminated 1, 2, 3, 4, 5, 6, 7, 8, 9, 10, 11, or 12 months after administration of TIMP-MG. In various embodiments, the use of alternative MG therapies is reduced or eliminated 1, 2, 3, 4, 5, 6, 7, 8, 9, 10, 11, or 12 years after administration of TIMP-MG.

The following analyses are contemplated to follow the immune status or tolerance induction of a subject receiving TIMP-MG therapy.
The proportion of antigen specific CD4+ T cells (CD154+CD137+ effector memory CD4+ T cells/total effector memory CD4+ T cells).
Proportion of activated antigen specific CD4+ T cells (CD38+CD154+CD137+ effector memory CD4+ T cells/CD154+CD137+ effector memory CD4+ T cells).
Proportion of antigen specific CD8+ T cells (CD154-CD137+CD8+ T cells/total CD8+ T cells).
Proportion of activated antigen specific CD8+ T cells (CD38+CD154-CD137+CD8+ T cells/CD154-CD137+CD8+ T cells).
Proportion of antigen specific T regulatory cells (CD154-CD137+CD127-CD25+TIGIT+ effector memory CD4+ T cells/CD154-CD137+ effector memory CD4+ T cells).
Change in proportion of anti-Acetylcholine Receptor (AChR) antibody levels (a positive result of >0.02 nmol/L is indicative of autoimmune myasthenia gravis)
Change in proportion of anti-Muscle Specific Kinase (Musk) antibody levels (a positive result of >0.02 nmol/L is indicative of autoimmune myasthenia gravis)

In various embodiments, the immune tolerance status of the subject determined prior to administration of TIMP-MG serves as the baseline. In various embodiments, the subject's baseline is determined from the assay of one or more biological samples 1, 2, 3, 4, 5, 6, or 7 days prior to administration of TIMP-MG. In various embodiments, the subject's baseline is determined from the assay of one or more biological samples 1, 2, 3, or 4 weeks prior to administration of TIMP-MG. In various embodiments, the subject's baseline is determined from the assay of one or more biological samples 1, 2, 3, 4, 5, 6, 7, 8, 9, 10, 11, or 12 months prior to administration of TIMP-MG.

In various embodiments, the immune tolerance status of the subject is determined after administration of TIMP-MG. In various embodiments, the immune tolerance status of the subject is determined from the assay of one or more biological samples 1, 2, 3, 4, 5, 6, or 7 days after administration of TIMP-MG. In various embodiments, the subject's Baseline is determined from the assay of one or more biological samples 1, 2, 3, or 4 weeks after administration of TIMP-MG. In various embodiments, the subject's baseline is determined from the assay of one or more biological samples 1, 2, 3, 4, 5, 6, 7, 8, 9, 10, 11, or 12 months after administration of TIMP-MG. In various embodiments, the subject's baseline is determined from the assay of one or more biological samples 1, 2, 3, 4, 5, 6, 7, 8, 9, 10, 11, or 12 years after administration of TIMP-MG. In various embodiments, the immune tolerance status of the subject determined after administration of TIMP-MG is compared to the baseline. In various embodiments, the immune tolerance status of the subject determined after the administration of TIMP-MG is compared to a healthy subject.

### Pharmaceutical Formulations

Pharmaceutical compositions of the present disclosure containing TIMP-MG described herein and an antigen may contain pharmaceutically acceptable carriers or additives depending on the route of administration. Examples of such carriers or additives include water, a pharmaceutically acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline®, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the inhibitors are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP-MG as described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### Kits

The disclosure also provides kits which comprise one or more compounds or compositions packaged in a manner which facilitates their use to practice methods of the disclosure. In one embodiment, such a kit includes a compound or composition described herein (e.g., a composition comprising a TIMP alone or in combination with another agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the particle compositions.

In a further embodiment, the disclosure provides an article of manufacture, or unit dose form, comprising: (a) a composition of matter comprising TIMP-MG as described herein; (b) a container containing said composition; and (c) a label affixed to said container, or a package insert included in said container referring to the use of said TIMP-MG in the treatment of MG as described herein.

Additional aspects and details of the disclosure will be apparent from the following examples, which are intended to be illustrative rather than limiting.

### EXAMPLES

### Example 1: CNP-106 Inhibits Antigen-Specific T Cell Responses in Animal Models

The efficacy of TIMP-MG (CNP-106) encapsulating AChR antigens with sequence SEQ ID NO: 3, SEQ ID NO: 5, SEQ ID NO: 8, SEQ ID NO: 13, SEQ ID NO: 19, SEQ ID NO: 20, SEQ ID NO: 21 (Table 2) at inducing tolerance was examined in a therapeutic model of MG. 8-11 week oldC57BL/6J mice were injected subcutaneously with Torpedo californica AChR plus human main immunogenic region (MIR) peptide on Day 0 (40 μg tAChR+100 μg MIR peptide in CFA), day 21, and day 56 (20 μg tAChR+100 μg MIR peptide in IFA). On day 42 and day 49 mice were injected intravenously with CNP-106 (1.25 mg/mouse or ~5 mg/kg HED and 0.5 mg/mouse or ~2 mg/kg HED). CNP encapsulating torpedo AChR (CNP-tAChR) was used as a positive control. Mice treated with saline were used as negative control.

Anti-AChR antibody titers were measured on day 28 and day 56. Grip test, clinical score, and EMG was evaluated on day 28 and day 70. On day 70, mice in each treatment group were sacrificed and their spleens harvested to assess T-cell responses.

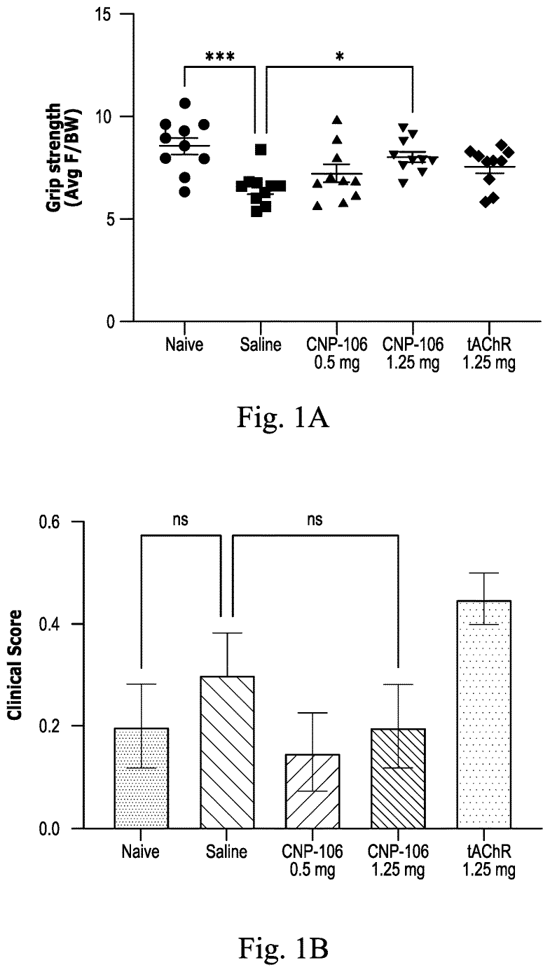

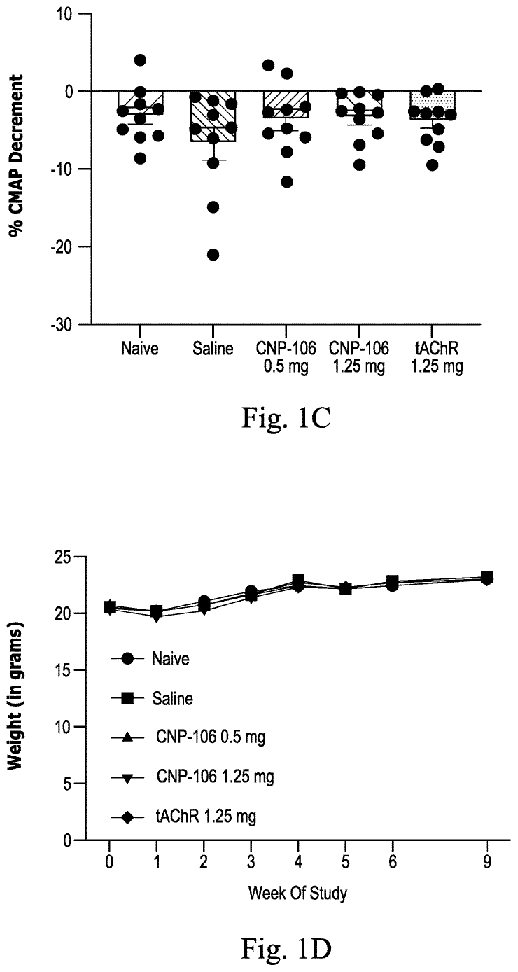

CNP-106 treatment (1.25 mg/dose) significantly improved grip strength compared to the saline treated group (FIG. 1A). (* p<0.05, *** p<0.0005)

CNP-106 treated mice also showed a trend towards improvement in overall clinical score when compared to the immunized saline control mice (FIG. 1B), electromyography (EMG) scores of mice from the various treatment groups represented as percent change in compound muscle action potential (CMAP) (FIG. 1C), and no weight change between the different treatment groups (FIG. 1D).

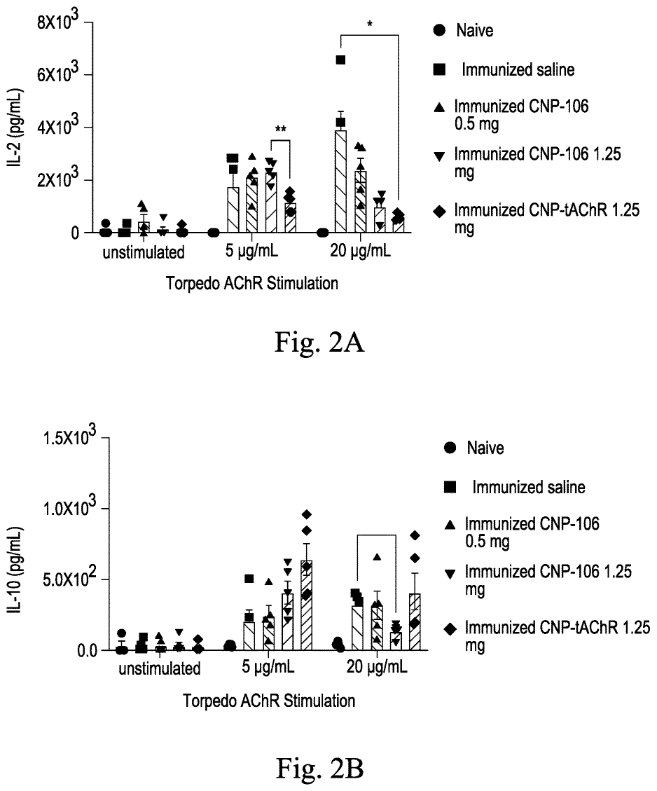

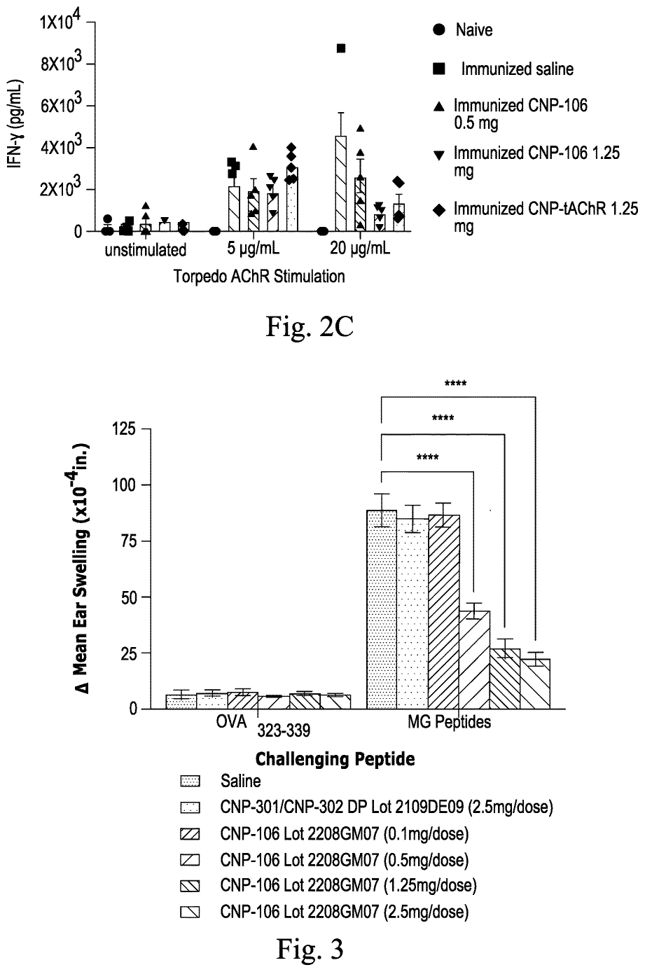

The AChR-specific T cell response was assessed via ex vivo recall responses from the spleen. Splenocytes from control and treated mice were cultured with media alone or with two different concentrations of tAChR (5 μg/mL and 20 μg/mL) and the level of IFN-γ, IL-2 and IL-10 secreted was assessed. CNP-106 treatment inhibits AChR specific T-cell responses. Treatment of mice immunized with 1.25 mg/dose of CNP-106 decreased the levels of IL-2 (FIG. 2A) (* p<0.05), IL-10 (FIG. 2B) (* p<0.05) and IFN-gamma (FIG. 2C).

Delayed-Type Hypersensitivity: C57BL/6 mice were primed with 100 μg (200 μL injection volume) of AChR-peptides emulsified in CFA on Day 0. On Day 0 and Day 7 post priming, mice were treated with CNP-106 administered via IV injection at a dose of 0.1 mg dose/house (0.4 mg/kg HED), 0.5 mg dose/mouse (2 mg/kg HED), 1.25 mg dose/mouse (5 mg/kg HED), 2.5 mg dose/mouse (10 mg/kg HED). On Day 14 post priming, the mice were challenged intradermally with 10 μg MG peptides (right ear, 10 μL injection volume) or Ovalbumin (left ear, 10 μL injection volume). The pinna thickness of each ear was measured immediately post-elicitation with MG peptides and OVA as well as 24 hours post elicitation. The change in pinna thickness (ΔT) for both ears of each mouse was calculated to assess the DTH response. CNP-106 significantly inhibited the DTH response compared to unloaded control particles at 0.5 mg dose/mouse (2 mg/kg HED), 1.25 mg dose/mouse (5 mg/kg HED), and 2.5 mg/dose (10 mg/kg HED) (FIG. 3) (**** p<0.00005).

### Example 2: Phase IB/IIA Trial of TIMP-MG

The present example describes a Phase 1b/2a randomized, double-blind, placebo-controlled clinical trial to assess the safety, tolerability, pharmacodynamics (PD), and efficacy of multiple ascending doses of CNP-106 in subjects with generalized MG. The study consists of a, Escalation Phase and an Expansion Phase with 42 days for Screening and 180 study days.

CNP-106 consists of PLGA particles encapsulating 7 AChRα and AChRε peptides (SEQ IDs NOs: 3, 5, 8, 13, 19, 20, 21). CNP-106 particles have an average diameter of 400-800 nm and a negative zeta potential of between −30 mV and −60 mV. CNP-106 particles are supplied as a lyophilized formulation. CNP-106 particles are reconstituted in sterile water for injection and diluted in sterile saline (0.9% sodium chloride) prior to administration.

Subjects ages 18-75 with generalized MG will be screened up to 42 days prior to enrollment into the study. If subjects are currently on a standard of care therapy, they will remain on their current standard of care therapy during the course of the study at the discretion of the Investigator regardless of treatment group (TIMP-MG or Placebo).

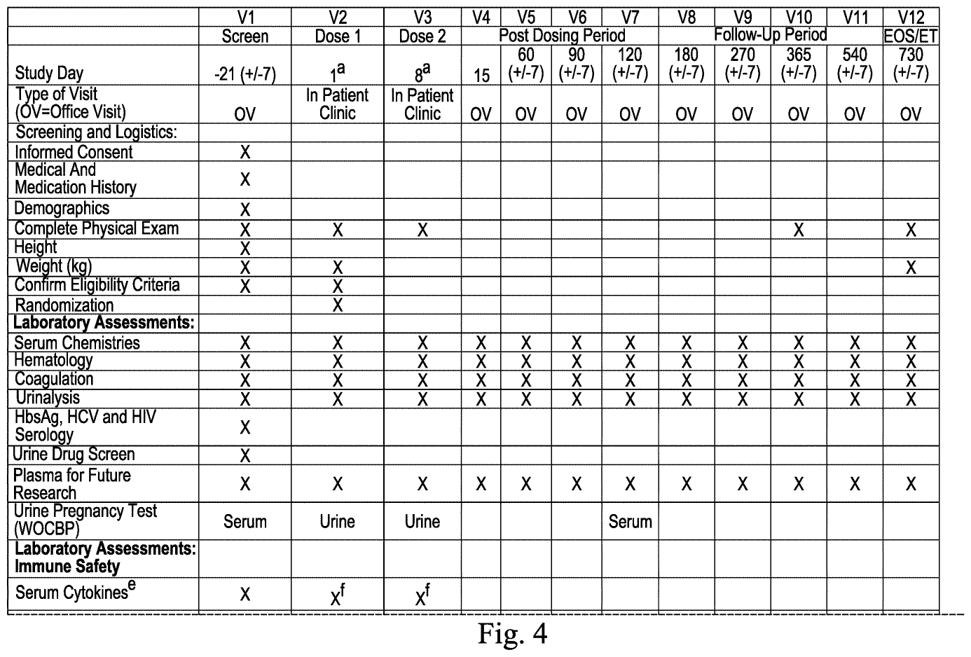

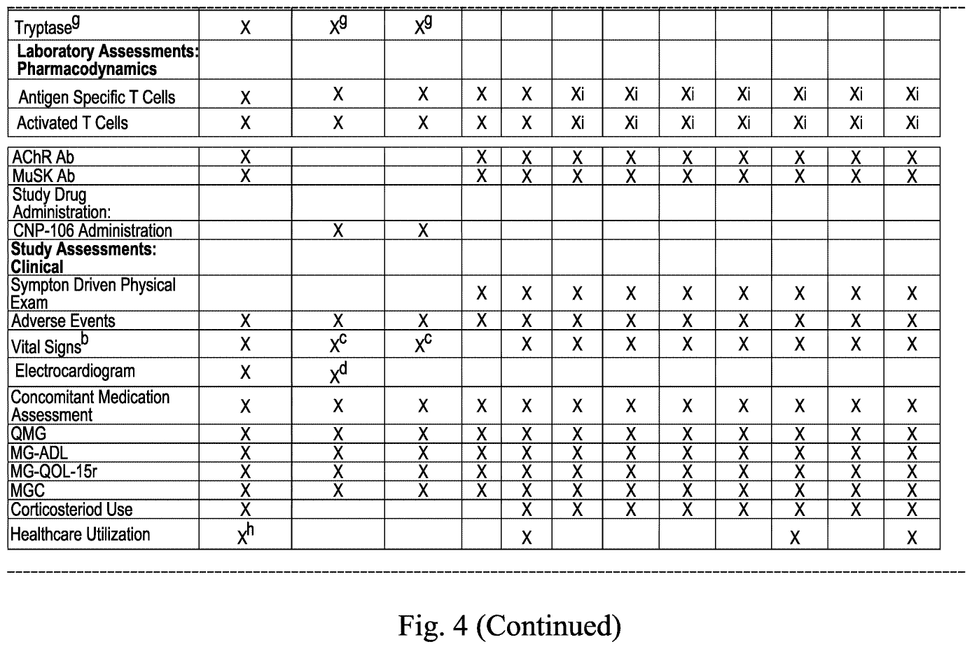

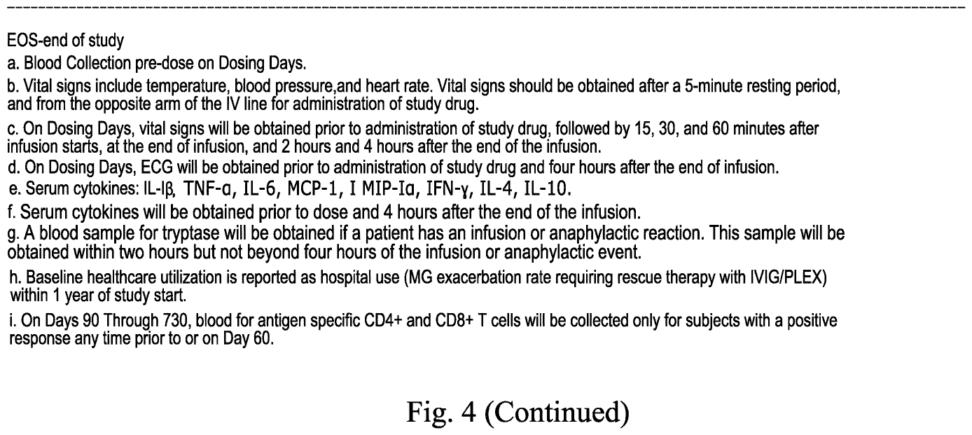

Screening will be completed per the schedule of events (FIG. 4) Subjects who meet all inclusion and no exclusion criteria after completing the Screen Visit will be enrolled in the study.

Subjects with generalized MG eligible for enrollment in this study are defined based on the following criteria:

1. Subjects who are willing and able to provide Institutional Review Board (IRB) approved written informed consent and privacy language as per national regulations.

2. Men and non-pregnant women, ages 18-75 years inclusive.

3. Female subjects of child-bearing potential must agree not to become pregnant during the study, have a negative pregnancy test at Screen Visit and agree to one of the following:
Use two highly effective forms of birth control starting at initial screening and continuing throughout the study duration.
Practice abstinence starting at initial screening and continuing through out the study duration.

4. Subjects with a Myasthenia Gravis Foundation of America Clinical Classification Class III-IV (Cohort 1). Upon successful Data Monitoring Committee (DMC) review and approval of preliminary safety data obtained from Cohort 1 through Day 15, Cohort 2 will enroll subjects with MGFA clinical classification class II-IV.

5. Subjects positive for anti-AChR antibodies by radioimmunoassay (RIA).

6. Subjects with MG-ADL Score≥6 at Screen and Baseline Visit with ≥50% of the score derived from non-ocular symptoms.

7. Subjects with QMG Score≥11 at Screen and Baseline Visit.

8. For subjects on steroids, subjects must agree not to change their steroid dose through study Day 60 unless reviewed and approved by the medical monitor and the site investigator.

9. For subjects on any medication used to treat the symptoms of MG (ex. Corticosteroids, pyridostigmine), subjects must be on a stable dose for a minimum of 90 days prior to enrollment and must agree not to increase their dose throughout the study duration unless reviewed and approved by the medical monitor and the site investigator.

10. Female subjects who agree to not breastfeed starting at initial screening and throughout study duration.

11. Female subjects who agree to not donate ova starting at initial screening and throughout study duration.

12. Male subjects with a spouse or partner of childbearing potential, who themselves and their spouse or partner agree to practice an effective form of birth control as discussed with their doctor by the study doctor or study staff starting at screening and throughout study duration.

Subjects will be randomized on Day 1 into the current Cohort in a 2:1 ratio to receive two separate administrations of intravenous CNP-106 or Placebo (0.9% sodium chloride injection USP) on Day 1 and Day 8. Investigational product will be administered by IV infusion over approximately 3-4 hours using a graduated rate of infusion. Subjects will undergo medical observation in the clinic for acute AEs for 4 hours following infusion on Day 1 and Day 8. Subjects will be discharged after 4 hours once all scheduled assessments for the visit day have been completed, if vital signs (sitting or supine blood pressure, heart rate, and body temperature) measured 4 hours post-infusion are within expected ranges for the subject and if no other health concerns are noted by the Investigator.

In the post-dosing period, subjects will return to the clinic for immune safety labs, PD measurements, QMG and MGC assessments, assessment of AEs, and medication changes per the Schedule of Events, (FIG. 4).

In the follow up period, subjects will return to the clinic for PD measurements, QMG and MGC assessments, assessments of AEs, and medication changes per schedule of events (FIG. 4).

Subjects will return to the clinic for the end of study visit for collection of safety labs (serum chemistries, hematology, coagulation, urinalysis), PD measurements, QMG and MGC assessments, and final assessment of AEs and medication changes.

The Escalation Phase is planned to enroll up to three cohorts (approximately 6 subjects per cohort) at multiple dose levels. Subjects will be randomized in a 2:1 ratio to receive either CNP-106 or Placebo (0.9% Sodium Chloride Injection USP) as a 200 mL intravenous infusion on Day 1 and Day 8. The planned dose level are as follows:

Cohort 1:150 mg

Cohort 2:350 mg

Cohort 3: Additional dose level as recommended by DMC

The Expansion Phase plans to enroll subjects randomized 2:1 to receive placebo or a safe and tolerable dose level of CNP-106 identified in the Escalation Phase. An additional cohort will be randomized to receive a third ( ) dose of CNP-106 on Day 90.

Subjects will receive CNP-106 as a 200 mL intravenous infusion on Day 1 and Day 8. Subjects randomized to receive a booster dose will receive CNP-106 as a 200 mL intravenous infusion on Day 1, Day 8 and Day 90. TIMP-MG will be administered using a graduated rate of infusion as follows: 20 mL/hr for first 15 min, 40 mL/hr, for next 15 min, 80 mL/hr for the remainder of the infusion.

Subjects will be assessed for safety, tolerability, PD and efficacy according to the schedule of events (FIG. 4) Primary objectives of this study include to assess the safety and tolerability of CNP-106. Primary endpoints include frequency of adverse events (AEs) and Serious Adverse Events (SAEs), MedDRA v, 25.0 or most current version, CTCAE v. 5.0 or most current version.

Exploratory endpoints of the study include change from baseline in Myasthenia Gravis Activities of Daily Living (MG-ADL) Score at Day 15, Day 60, Day 90, Day 120, Day 180, (optionally Day 365, Day 540, and Day 730); change from baseline in Quantitative Myasthenia Gravis (QMG) Score at Day 15, Day 60, Day 90, Day 120, Day 180 (optionally Day 365, Day 540, and Day 730); change from baseline in Myasthenia Gravis Quality of Life 15-revised (MG-QOL15r) Score at Day 15, Day 60, Day 90, Day 120, Day 180 (optionally Day 365, Day 540, and Day 730); change from baseline in health care utilization by hospital visits (MG exacerbation rate requiring rescue therapy with IVIG/PLEX) at Day 60, Day 90, Day 180 (optionally Day 365, Day 540, and Day 730); change from baseline in Myasthenia Gravis Composite (MGC) Score at Day 15, Day 60, Day 90, Day 120, Day 180 (optionally Day 365, Day 540, and Day 730); change from baseline in antigen specific (number or levels) CD4+ and CD8+ T cells in PBMC at Day 15, Day 60, Day 90, Day 120, Day 180 (optionally Day 365, Day 540, and Day 730); change from baseline in activated antigen specific (number or levels) CD4+ and CD8+ T cells in PBMC at Day 15, Day 60, Day 90, Day 120, Day 180 (optionally Day 365, Day 540, and Day 730); change from baseline in anti-Acetylcholine Receptor (AChR) antibody levels at Day 15, Day 60, Day 90, Day 120, Day 180 (optionally Day 365, Day 540, and Day 730); change from baseline in anti-Muscle Specific Kinase (Musk) antibody levels at Day 15, Day 60, Day 90, Day 120, Day 180 (optionally Day 365, Day 540, and Day 730).

Clinical efficacy endpoints include MG-ADL score, QMG score, MG-QOL-15r score, Healthcare utilization (MG exacerbation rate requiring rescue therapy with IVIG/PLEX), MGC score.

The total duration of the study is up to 222 days for the primary study (42 days for Screening, 180 Study Days) and optionally 508 days for the long-term safety follow-up period.

Safety Assessments include:
1. Complete physical examination (PE) consisting of the following systems on Screening day, day 1, and day 8: cardiovascular; dermatological; ear, nose, and throat; extremities; gastrointestinal; musculoskeletal; ophthalmological; neurological; respiratory. Height and weight will be obtained at screening.

2. Vital signs at Screening, Day 1, day 8, day 60, day 90, day 120, day 180, day 270, day 365, day 540, and day 730.

3. Electrocardiogram on Screening, day 1 and day 8.

4. AEs at Screen followed by day1, day 8, day 15, day 60, day 90, day 120, day 180, day 270, day 365 day 540, and day 730.

5. Safety laboratory tests (serum chemistry, hematology, coagulation and urinalysis) at Screen followed by day 1, 8, 15, 60, 90, 120, 180, 180, 270, 365, 540 and 730.

6. Cytokine (IL-1B, TNF-α, IL-6, MCP-1, MIP-1α, IFN-γ, IL-4, IL-10) and tryptase profiling at Screen followed by Days 1, 8.

Laboratory/PD assessments include:

1. Antigen specific CD4+ and CD8+T-cells at Screen followed by day1, day 8, day 15, day 60, day 90, day 120, day 180, day 270, day 365, day 540, and day 730.

2. Activated antigen specific CD4+ and CD8+T-cells at Screen followed by day1, day 8, day 15, day 60, day 90, day 120, day 180, day 270, day 365, day 540, and day 730.

3. Anti-AChR antibodies and anti-MuSK antibodies at Screening followed by day1, day 15, day 60, day 90, day 120, day 180, day 270, day 365, day 540, and day 730.

Clinical efficacy assessments will include:

1. Symptom driven physical exam on day 15, day 60, day 90, day 120, day 180, day 270, day 365, day 540, and day 730.

4. QMG, MG-ADL, MG-QOL-15r, MGC scores at Screening followed by day1, day 8, day 15, day 60, day 90, day 120, day 180, day 270, day 365, day 540, and day 730.

2. Steroid use (mean daily prednisone) on day 60, day 90, day 120, day 180, day 270, day 365, day 540, and day 730.

3. Healthcare utilization at Screening, day 60, Day 90, Day 120, Day 180, day 365 and day 730 reported as hospital use (MG exacerbation rate requiring rescue therapy with IVIG/PLEX) within 1 year of study start.

### Example 3. Patient Selection Criteria for CNP-106 Treatment

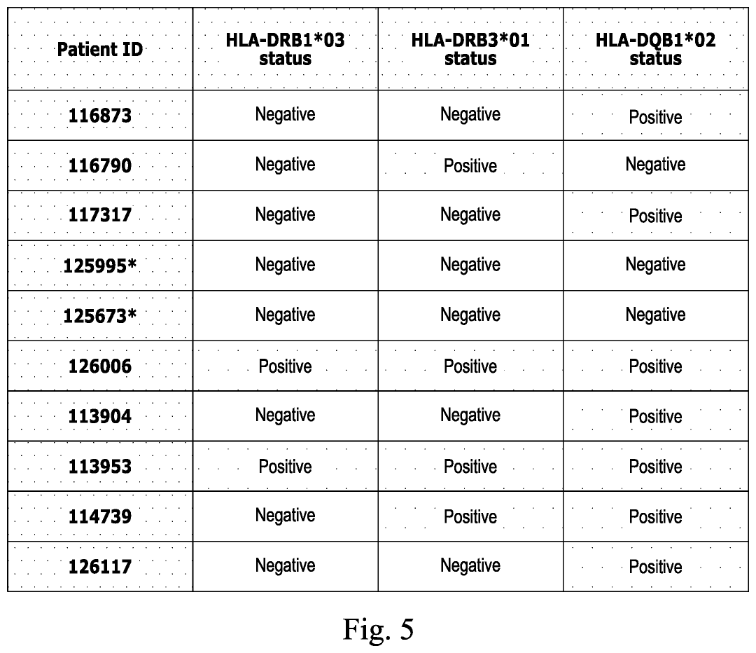

PBMCs were obtained from 10 MG patients to determine if there was a correlation with AChR restricted HLA positivity. Since CNP-106 encapsulates AChR antigens, subjects with HLA-DRB1*03, HLA-DRB3*01, HLA-DQB1*02 status can be a criteria for inclusion in the CNP-106 clinical trial. 80% of patients in the study expressed an HLA restriction, HLA-DRB1*03, HLA-DRB3*01, HLA-DQB1*02 for AChR (FIG. 5). Selection criteria could identify patients likely to achieve efficacy with CNP-105. MG patients whose disease is driven by autoreactivity to the AChR antigens are eligible for treatment with CNP-106 and can be identified based on HLA haplotype in blood.

### Example 4. Process for Preparing MG Peptide Solution for the Manufacture of Tolerizing Nanoparticles Encapsulating MG Associated Antigens (TIMP-MG)

Acid soluble AChR antigen(s) ((AChRα (43-58), AChRα (68-113), AChRα (121-158), AChRα isoform a (324-357), AChRe (201-250) and AChRe (414-435)) were dissolved in 0.5N acetic acid with an individual AChR Peptide concentration of 2 mg/mL.

Base soluble AChR antigen(s) (AChRe (116-130)) are dissolved in 0.1% ammonium hydroxide with an individual peptide concentration of 8 mg/mL.

The acid and base dissolved antigens are sterile filtered using 0.2 μm nylon syringe filters. The filtered 6-peptide solution in 0.5N acetic acid and the filtered 1-peptide solution in 0.1% ammonium hydroxide are combined at 4:1 ratio with a final individual peptide concentration of 1.6 mg/mL.

### Example 5. Process for Manufacturing Tolerizing Nanoparticles Encapsulating MG Associated Antigens (TIMP-MG)

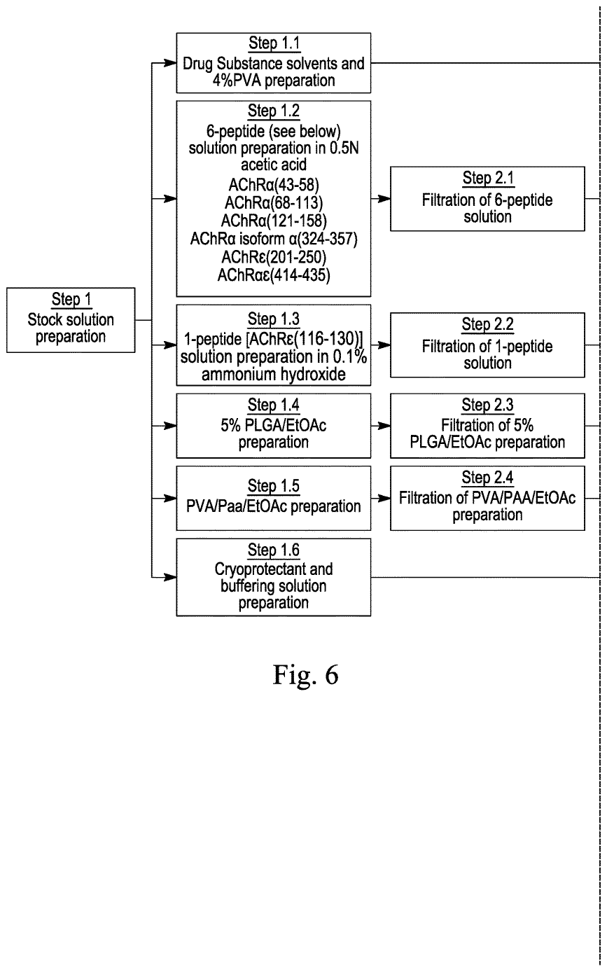

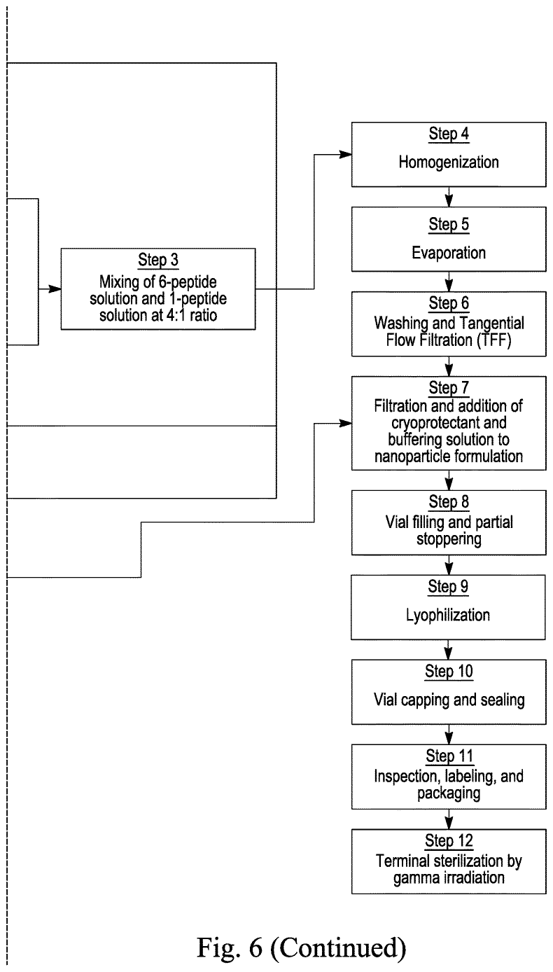

TIMP-MG (TIMP-MG) was manufactured using a double-emulsion solvent evaporation process. A high-level manufacturing process flow diagram is shown in FIG. 6. Briefly, the filtered peptide mixture is rapidly mixed with a 5% PLGA solution (50:50; molecular weight between 10,000 to 60,000 Da) in ethyl acetate to generate a primary water-in-oil emulsion. PVA (4% in water), PAA (Sigma Aldrich, 100 kDa, 35% wt) and ethyl acetate were admixed to generate a blend. The composition of PVA/PAA/ethyl acetate blend is maintained at a pH below 4.0. The primary emulsion was then mixed with the PVA/PAA/ethyl acetate blend to form an oil-in-water secondary emulsion. Mixing of the primary and secondary emulsions was performed by homogenization.

Solvent was removed from the secondary emulsion by evaporation under pressure for a total of at least 3-4 hours. Hardened nanoparticles were then washed in sterile water and concentrated by filtration using a 20 μm filter. Cryoprotectants sucrose and mannitol and buffering agent sodium citrate were added to the hardened nanoparticles. The formulation was then lyophilized.

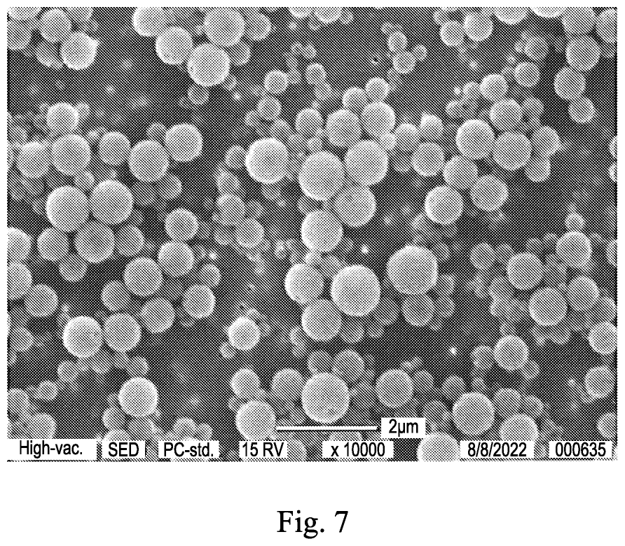

The final TIMP-MG formulation was characterized to determine physiochemical properties such as particle diameter, zeta potential, size distribution, and total peptide content. The results of TIMP-MG characterization are provided in Table 4. TIMP-MG particles were examined by Scanning Electron Microscopy showing a homogenous composition of intact particles with smooth surfaces (FIG. 7).

TABLE 4
Physiochemical characterization of CNP-106 particles

| NANOPARTICLE PHYSIOCHEMICAL PROPERTY | RESULT | |
|---|---|---|
| Particle size by dynamic light scattering | 570 | nm |
| Zeta potential by dynamic light scattering | −50 | mV |
| Particle size by laser diffraction | 586 | nm |
| Size distribution by single particle optical | 520/570/660 | |
| sensing (SPOS) | (d10/d50/d90) | |
| RP-HPLC Content - AChRα (43-58) | 1.3 | μg/mg |
| RP-HPLC Content - AChRα (68-113) | 1.6 | μg/mg |
| RP-HPLC Content - AChRα (121-158) | 1.1 | μg/mg |
| RP-HPLC Content - AChRα isoform A | 1.5 | μg/mg |
| (324-357) | | |
| RP-HPLC Content - AChRε (116-130) | 1.3 | μg/mg |
| RP-HPLC Content - AChRε (201-250) | 1.4 | μg/mg |
| RP-HPLC Content - AChRε (414-435) | 0.9 | μg/mg |
| Endotoxin | <0.048 | EU/mg |

### Example 6 Pharmaceutical Compositions of TIMP-MG

Pharmaceutical formulations or compositions of TIMP-MG comprise individual components manufactured at the 80 g and 160 g batch scale listed in Table 5.

TABLE 5

| COMPONENT | 80 G BATCH | 160 G BATCH |
|---|---|---|
| PLGA nanoparticles | 30-40% | 30-40% |
| Sucrose | 30-40% | 30-40% |
| Mannitol | 20-30% | 20-30% |
| Sodium citrate | 1-10% | 1-10% |
| MG associated antigens | 0.1 μg/mg of PLGA | 0.1 μg/mg of PLGA |
| | to 2.6 μg/mg of | to 2.6 μg/mg of |
| | PLGA | PLGA |
| RP-HPLC content - AChRα | 0.1 μg/mg of PLGA | 0.1 μg/mg of PLGA |
| (43-58) | to 2.6 μg/mg of | to 2.6 μg/mg of |
| | PLGA | PLGA |
| RP-HPLC content - AChRα | 0.1 μg/mg of PLGA | 0.1 μg/mg of PLGA |
| (68-113) | to 2.6 μg/mg of | to 2.6 μg/mg of |
| | PLGA | PLGA |
| RP-HPLC content - AChRα | 0.1 μg/mg of PLGA | 0.1 μg/mg of PLGA |
| (121-158) | to 2.6 μg/mg of | to 2.6 μg/mg of |
| | PLGA | PLGA |
| RP-HPLC content - AChRα | 0.1 μg/mg of PLGA | 0.1 μg/mg of PLGA |
| ISOFORM A (324-357) | to 2.6 μg/mg of | to 2.6 μg/mg of |
| | PLGA | PLGA |
| RP-HPLC content - AChRε | 0.1 μg/mg of PLGA | 0.1 μg/mg of PLGA |
| (116-130) | to 2.6 μg/mg of | to 2.6 μg/mg of |
| | PLGA | PLGA |
| RP-HPLC content - AChRε | 0.1 μg/mg of PLGA | 0.1 μg/mg of PLGA |
| (201-250) | to 2.6 μg/mg of | to 2.6 μg/mg of |
| | PLGA | PLGA |
| RP-HPLC content - AChRε | 0.1 μg/mg of PLGA | 0.1 μg/mg of PLGA |
| (414-435) | to 2.6 μg/mg of | to 2.6 μg/mg of |
| | PLGA | PLGA |

### Example 7. Process for Preparing MG Antigens for the Manufacture of TIMP-MG

Acid soluble AChR antigen(s) ((AChRα (43-58), AChRα (68-113), AChRα (121-158), AChRα isoform a (324-357), AChRc (201-250) and AChRe (414-435)) will be dissolved in 0.5N acetic acid containing 0.1% LMPG detergent.

Base soluble AChR antigen(s) (AChRe (116-130)) will be dissolved in 0.1% ammonium hydroxide containing 0.1% LMPG detergent.

The dissolved antigens will be sterile filtered using 0.2 μm nylon syringe filters. The filtered 6-peptide solution in 0.5N acetic acid and 0.1% LMPG and the filtered 1-peptide solution in 0.1% ammonium hydroxide containing 0.1% LMPG will be combined at 4:1 ratio. The antigens will then be rapidly mixed with a 5% PLGA solution (50:50; molecular weight between 10,000 to 60,000 Da) in ethyl acetate to generate a primary water-in-oil emulsion. PVA (4% in water), PAA (Sigma Aldrich, 100 kDa, 35% wt) and ethyl acetate will be admixed to generate a blend. The composition of PVA/PAA/ethyl acetate blend will be maintained at a pH below 4.0. The primary emulsion will be mixed with the PVA/PAA/ethyl acetate blend to form an oil-in-water secondary emulsion. Mixing of the primary and secondary emulsions will be performed by homogenization.

Solvent will be removed from the secondary emulsion by evaporation under pressure for a total of at least 3-4 hours. Hardened nanoparticles will be washed in sterile water and concentrated by filtration using a 20 μm filter. Cryoprotectants sucrose and mannitol and buffering agent sodium citrate dihydrate will be added to the hardened nanoparticles. The formulation will be lyophilized.

The final TIMP-MG formulation will be characterized to determine physiochemical properties such as particle diameter, zeta potential, total peptide content.

It is understood, therefore, that this invention is not limited to the particular embodiments disclosed, but is intended to cover all modifications which are within the spirit and scope of the invention as defined by the appended claims; the above description, and/or shown in the attached drawings. Consequently, only such limitations as appear in the appended claims should be placed on the disclosure.

### REFERENCES

1. Conti-Fine, B. M., Milani, M., & Kaminski, H. J. Myasthenia gravis: past, present, and future. The Journal of clinical investigation, 116 (11), 2843-2854 (2006).
2. Wang, Z. Y., Karachunski, P. I., Howard, J. F., Jr. & Conti-Fine, B. M. Myasthenia in SCID mice grafted with myasthenic patient lymphocytes: role of CD4+ and CD8+ cells. Neurology 52, 484-497 (1999).
3. Gilhus, N. E., Skeie, G. O., Romi, F., Lazaridis, K., Zisimopoulou, P., & Tzartos, S. Myasthenia gravis-autoantibody characteristics and their implications for therapy. Nature reviews. Neurology, 12 (5), 259-268 (2016).
4. Carr, A. S., Cardwell, C. R., McCarron, P. O. et al. A systematic review of population based epidemiological studies in Myasthenia Gravis. BMC Neurol 10, 46 (2010)
5. Meliani A, Boisgerault F, Hardet R, et al. Antigen-selective modulation of AAV immunogenicity with tolerogenic rapamycin nanoparticles enables successful vector re-administration. Nat Commun, 9 (1): 4098 (2018).
6. Farmakidis, C., Pasnoor, M., Dimachkie, M. M., & Barohn, R. J. Treatment of Myasthenia Gravis. Neurologic clinics, 36 (2), 311-337 (2018).
7. Barnett, C., Herbelin, L., Dimachkie, M. M., & Barohn, R. J. (2018). Measuring Clinical Treatment Response in Myasthenia Gravis. Neurologic clinics, 36 (2), 339-353.
8. Getts D R, Martin A J, McCarthy D P, et al. Microparticles bearing encephalitogenic peptides induce T-cell tolerance and ameliorate experimental autoimmune encephalomyelitis. Nature Biotechnology. 2012; 30 (12): 1217-1224.
9. Hunter Z, McCarthy D P, Yap W T, et al. A biodegradable nanoparticle platform for the induction of antigen-specific immune tolerance for treatment of autoimmune disease. ACS Nano. 2014; 8 (3): 2148-2160.
10. Kelly C P, Murray J A, Leffler D A, et al. TAK-101 Nanoparticles Induce Gluten-Specific Tolerance in Celiac Disease: A Randomized, Double-Blind, Placebo-Controlled Study. Gastroenterology. 2021; 161 (1): 66-80.e68.
11. Prasad S, Neef T, Xu D, et al. Tolerogenic Ag-PLG nanoparticles induce tregs to suppress activated diabetogenic CD4 and CD8 T cells. J Autoimmun. 2018; 89:112-124.
12. Smarr C B, Yap W T, Neef T P, et al. Biodegradable antigen-associated PLG nanoparticles tolerize Th2-mediated allergic airway inflammation pre- and postsensitization. Proc Natl Acad Sci USA. 2016; 113 (18): 5059-5064.
13. Bilati U, Allemann E, Doelker E. Development of a nanoprecipitation method intended for the entrapment of hydrophilic drugs into nanoparticles. Eur J Pharm Sci. 2005 January; 24 (1): 67-75.
14. Mejía S P, Sánchez A, Vásquez V, Orozco J. Functional Nanocarriers for Delivering Itraconazole Against Fungal Intracellular Infections. Front Pharmacol. 2021 Jun. 28; 12:685391.
15. Zielińska A, Carreiró F, Oliveira A M, Neves A, Pires B, Venkatesh D N, Durazzo A, Lucarini M, Eder P, Silva A M, Santini A, Souto E B. Polymeric Nanoparticles: Production, Characterization, Toxicology and Ecotoxicology. Molecules. 2020 Aug. 15; 25 (16): 3731.

We claim:

1. A composition comprising tolerizing immune modifying particle-Myasthenia Gravis (TIMP-MG) particles encapsulating one or more Myasthenia Gravis (MG) associated antigens, portions thereof, or combinations thereof.

2. The composition of claim 1, wherein the particles comprise a biodegradable polymer.

3. The composition of claims 1-2, wherein the biodegradable polymer is polyglycolic acid (PGA), poly(lactide-co-glycolide) (PLG), polylactic acid (PLA), a co-polymer of PLG and PLA (PLGA), polycaprolactone (PCL), polystyrene, polysebacic acid (PSA), poly(lactic-co-sebacic) acid (PLSA), polypropylene sulfide, cyclodextran, poly ethylene glycol (PEG), chitosan, a polysaccharide, one or more lipids, a liposome, diamond, iron, zinc, cadmium, gold or silver.

4. The composition of any one of claims 1-3, wherein the particles have a negative zeta potential.

5. The composition of claim 4, wherein the particles have a zeta potential between −1 and −100 mV.

6. The composition of claim 4 or 5, wherein the particles have a zeta potential between −30 and −80 mV.

7. The composition of any one of claims 1-6, wherein the particle size is between 100 nm and 1000 nm.

8. The composition of claim 7, wherein the particle size is between 400-800 nm.

9. The composition of any one of claims 1-8, wherein the antigen comprises one or more proteins, peptides, or one or more antigenic epitopes thereof.

10. The composition of claim 9, wherein the antigen is the AChR protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3) and/or a portion thereof, or one or more antigenic epitopes thereof.

11. The composition of claim 9, wherein the antigen is an MG associated antigen as set out in Table 1.

12. The composition of claim 9, wherein the antigen is the AChR protein subunit and/or a portion thereof, or one or more antigenic epitopes thereof.

13. The composition of claim 12, wherein the AChR antigenic epitopes are selected from the group consisting of SEQ ID NOs: 1 to 21.

14. The composition of claim 13, wherein the AChR antigenic epitopes comprise a combination of SEQ ID NO: 3, SEQ ID NO: 5, SEQ ID NO: 8, SEQ ID NO: 13, SEQ ID NO: 19, SEQ ID NO: 20, and/or SEQ ID NO: 21.

15. A method of inducing tolerance in a subject in need thereof comprising administering to the subject a composition comprising negatively charged particles encapsulating an antigen, wherein the antigen is one or more Myasthenia Gravis (MG) associated antigens, portions thereof, or combinations thereof.

16. The method of claim 15, wherein the particle comprises polyglycolic acid (PGA), poly(lactide-co-glycolide) (PLG), polylactic acid (PLA), a co-polymer of PLG and PLA (PLGA), polycaprolactone (PCL), polystyrene, polysebacic acid (PSA), poly(lactic-co-sebacic) acid (PLSA), polypropylene sulfide, cyclodextran, poly ethylene glycol (PEG), chitosan, a polysaccharide, one or more lipids, a liposome, diamond, iron, zinc, cadmium, gold or silver.

17. The method of any one of claims 15-16, wherein the particles have a negative zeta potential.

18. The method of any one of claims 15-17, wherein the particles have a zeta potential between 0 mV and −100 mV.

19. The method of any one of claims 15-18, wherein the particles have a zeta potential between −30 mV and −100 mV.

20. The method of any one of claims 15-19, wherein the size of the particle is between 100 nm and 1000 nm.

21. The method of any one of claims 15-20, wherein the size of the particle is between 400 nm and 800 nm.

22. The method of claim of any one of claims 15-21, wherein the antigen comprises one or more proteins, peptides or one or more antigenic epitopes thereof.

23. The method of claim 22, wherein the antigen is the AChR protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3) and/or a portion thereof, or one or more antigenic epitopes thereof.

24. The method of claim 22, wherein the antigen is an MG associated antigen set out in Table 1.

25. The method of claim 22, wherein the antigen is the AChR protein subunit and/or a portion thereof, or one or more antigenic epitopes thereof.

26. The method of claim 25, wherein the AChR antigenic epitopes are selected from the group consisting of SEQ ID NOs: 1 to 21.

27. The method of claim 26, wherein the AChR antigenic epitopes comprise a combination of SEQ ID NO: 3, SEQ ID NO: 5, SEQ ID NO: 8, SEQ ID NO: 13, SEQ ID NO: 19, SEQ ID NO: 20, and/or SEQ ID NO: 21.

28. The method of any one of claims 15-27, wherein the subject has an autoimmune disease.

29. The method of any one of claims 15-28, wherein the autoimmune disease is Myasthenia Gravis.

30. The method of any one of claims 15-29, wherein administration of the said composition reduces an MG associated immune response.

31. The method of claim 30, wherein the immune response is an inflammatory immune response.

32. The method of any one of claims 30-31, wherein the immune response is humoral immune response.

33. The method of any one of claims 30-31, wherein the immune response is an adaptive immune response.

34. The method of any one of claims 30-31, wherein the immune response is an innate immune response.

35. The method of any one of claims 30-34, wherein the immune response is a T cell, B cell, monocyte, macrophage, neutrophil, basophil, or eosinophil response.

36. The method of any one of claims 30-34, wherein the immune response is an antibody response.

37. The method of claim 36, wherein the antibody response is the formation of AChR antibodies against the AChR protein, antigens, epitopes or portions thereof.

38. The method of claim 36, wherein the antibody response is the formation of MuSK antibodies against the MuSK protein, antigens, epitopes or portions thereof.

39. The method of any one of claims 15-38, wherein the composition is administered intravenously, intramuscularly, ocularly, intraperitoneally, transdermally, nasally, orally and/or subcutaneously.

40. A method of treating Myasthenia Gravis (MG) in a subject comprising administering to the subject tolerizing immune modifying particles encapsulating one or more MG associated antigens (TIMP-MG).

41. The method of claim 40, wherein the MG associated antigen is the AChR protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3) and/or a portion thereof, or one or more antigenic epitopes thereof.

42. The method of claim 40 or 41, wherein the antigen is an MG associated antigen selected from the group comprising table 1.

43. The method of any one of claims 40-42, wherein the antigen is the AChR protein subunit and/or a portion thereof, or one or more antigenic epitopes thereof.

44. The method of any one of claim 40-43 wherein the AChR antigenic epitopes are selected from the group consisting of SEQ ID NOs: 1 to 21.

45. The method of any one of claims 43-44, wherein the AChR antigenic epitopes comprise a combination of SEQ ID NO: 3, SEQ ID NO: 5, SEQ ID NO: 8, SEQ ID NO: 13, SEQ ID NO: 19, SEQ ID NO: 20, SEQ ID NO: 21.

46. The method of any one of claims 40-45, wherein the TIMP-MG particles have an average diameter of between 100 nm and 1000 nm.

47. The method of claim 46, wherein the TIMP-MG particles have an average diameter of between 400 nm and 800 nm.

48. The method of any one of claim 40-47, wherein the TIMP-MG particles have a negative zeta potential.

49. The method of claim 48, wherein the particles have a negative zeta potential of between 0 mV and −100 mV.

50. The method of claim 49, wherein the particles have a negative zeta potential of between-30 mV and −80 mV.

51. The method of any one of claims 40-50, wherein TIMP-MG is administered at a concentration between 0.0005 mg/mL and 50 mg/mL.

52. The method of any one of any one of claims 40-50, wherein TIMP-MG is administered at a dose level of 0.1 mg/kg, 0.25 mg/kg, 0.5 mg/kg, 1.0 mg/kg, 2.0 mg/kg, 4.0 mg/kg, 6 mg/kg, 8.0 mg/kg, 10 mg/kg, or 12 mg/kg.

53. The method of any one of any one of claims 40-50, wherein TIMP-MG is administered at a dose level of between 0.1 mg and 800 mg.

54. The method of claim 53, wherein TIMP-MG is administered at a dose of 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

55. The method of any one of claims 40-54, wherein TIMP-MG is administered in a single dose or in multiple doses.

56. The method of any one of any one of claims 40-55, wherein TIMP-MG is administered once weekly, once every two weeks, once every three weeks, once every 4 weeks, once every two months, once every three months, once every 6 months, or once per year.

57. The method of any one of any one of claims 40-56, wherein TIMP-MG is administered in two doses one week apart.

58. The method of any one of any one of claims 40-56, wherein TIMP-MG is administered in two doses one week apart followed by a booster dose administered as a single dose once every three months.

59. The method of claims 40-58, wherein TIMP-MG is administered intravenously, subcutaneously, intramuscular, intraperitoneally, intranasally, or orally.

60. The method of any one of claims 15-59, wherein administering TIMP-MG to a subject reduces or ameliorates one or more symptoms of MG.

61. The method of claim 60, wherein the one or more symptoms of MG are selected from the group consisting of droopy eyelids (ptosis), double vision (diplopia), difficulty making facial expressions, problems chewing, difficulty swallowing, slurred speech (dysarthria), shortness of breath, breathing difficulties, muscle fatigue, skeletal muscle weakness, neck weakness, limb weakness, dysphagia, dysphonia, weakened neuromuscular junctions, impaired synaptic signal transduction, muscle damage, anti-Musk antibodies, anti-AChR antibodies, increased activated CD4+ cells in PBMCs, and increased activated CD8+ T-cells in PBMCs, increased Myasthenia Gravis Activities of Daily Living Score, increased Quantitative MG Score, MG Quality of Life 15-revised score, and MG Composite Score.

62. The method of any one of claims 40-61, wherein administering TIMP-MG in a subject reduces duration and/or severity of an inflammatory immune response to MG antigens.

63. The method of claim 62, wherein the inflammatory immune response is a T cell, B cell, or a myeloid cell response.

64. The method of claims 62-63, wherein the inflammatory immune response is assayed from one or more biological samples obtained from the subject.

65. The method of claim 64, wherein the biological sample is selected from the group consisting of whole-blood, peripheral blood, peripheral blood mononuclear cells (PBMCs), serum, plasma, urine, cerebrospinal fluid (CSF), stool, a tissue biopsy, and/or a bone-marrow biopsy.

66. The method of any one of claims 40-65, wherein administering TIMP-MG in a subject reduces levels of antigen-specific CD4+ and CD8+ T cells.

67. The method of claim 66, wherein administering TIMP-MG decreases the levels of antigen specific CD4+ and/or CD8+ T cells by 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values).

68. The method of any one of claims 40-65, wherein administering TIMP-MG in a subject reduces levels of activated antigen-specific CD4+ and CD8+ T cells.

69. The method of claim 68, wherein treatment with TIMP-MG decreases the levels of activated antigen specific CD4+ and/or CD8+ T cells by 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values)

70. The method of any one of claims 40-65, wherein administering TIMP-MG in a subject increases levels of antigen specific T regulatory cells.

71. The method of any one of claims 40-65, wherein administering TIMP-MG in a subject reduces levels of anti-AChR antibodies.

72. The method of claim 71, wherein treatment with TIMP-MG decreases the levels of anti-AChR antibodies by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values)

73. The method of any one of claims 40-65, wherein administering TIMP-MG in a subject reduces levels of anti-MuSK antibodies.

74. The method of claim 73, wherein treatment with TIMP-MG decreases the levels of anti-MuSK antibodies by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values).

75. The method of claim 40, wherein treatment with TIMP-MG decreases health care utilization by hospital visits, decreases MG exacerbation rate, or rescue therapy with IVIG/PLEX.

76. The method of claim 40, wherein treatment with TIMP-MG decreases use of alternative MG therapies.

77. The method of claim 40, wherein amelioration in one or more symptoms of MG is determined using MG-ADL Score, QMG Score, GM-QOL-15r Score, Healthcare Utilization (MG exacerbation rate requiring rescue therapy with IVIG/IPLEX), and/or MGC Score.

78. The method of claim 40, wherein TIMP-MG administration is based on the subject's genetic profile/HLA haplotype, Myasthenia Gravis Foundation of America (MGFA) clinical classification score, baseline MG-ADL score, baseline QMG score, baseline MGC score, symptoms, concurrent or previously administered medications/therapeutics/drugs, concurrent or active diseases, presence/levels of anti-AChR antibodies, presence/levels of anti-MuSK antibodies or other MG associated antibodies.

79. A method for preparing a composition comprising particles encapsulating one or more MG associated antigens, the method comprising:
(a) generating an aqueous solution of one or more MG associated antigens;
(b) generating a primary emulsion by mixing an aqueous solution of step (a) with an oil phase including a polymer;
(c) mixing the primary emulsion with a mixture comprising one or more surfactants and/or stabilizers to form a secondary emulsion; and
(d) hardening the secondary emulsion of step (c) by evaporation resulting in hardened polymeric nanoparticles encapsulating MG associated antigens within their cores.

80. The method of claim 79, further comprising (e) filtering, washing, and concentrating the nanoparticles.

81. The method of claim 79 or 80 further comprising (f) freeze drying the nanoparticle.

82. The method of claim 79, wherein the aqueous solution of step (a) includes a solvent.

83. The method of claim 80 or 81, wherein the solvent of step (a) is selected from the group comprising acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid, barium hydroxide, calcium hydroxide, chromium hydroxide, potassium hydroxide, ammonium hydroxide, zinc hydroxide, barium hydroxide, sodium bicarbonate, methylamine, diethylamine, sodium hydroxide, magnesium hydroxide, ammonium bicarbonate, ammonia, aluminium hydroxide, sodium carbonate, magnesium hydroxide, zinc hydroxide, ferrous hydroxide, acetone, lithium hydroxide, pyridine, rubidium hydroxide.

84. The method of any one of claims 82-83, wherein one or more MG associated antigens in step (a) may be dissolved in the same solvent or different solvent.

85. The method of claims 82-84, wherein tone or more MG associated antigens in step (a) are dissolved in acid or base.

86. The method of claim 85, wherein the one or more MG associated antigens dissolved in acid and base are admixed at a ratio of 100:1, 50:1, 25:1, 20:1, 10:1, 9:1, 8:1, 7:1, 6:1, 5:1, 4:1, 3:1, 2:1 or 1:1.

87. The method of any one of claims 79-86, wherein the mixtures of step (b) and step (c) include one or more solvents.

88. The method of claim 87, wherein the solvent is an organic solvent or an inorganic solvent.

89. The method of any one of claims 87-88, wherein the solvents are the same.

90. The method of any one of claims 87-88, wherein the solvents are different.

91. The method of any one of claims 88-90 wherein the organic solvent is selected from the group comprising acetone, ethanol, methylene chloride (dichloromethane), dimethyl sulfoxide (DMSO), ethyl acetate, dimethylformamide, tetrahydrofuran, chloroform, and acetic acid.

92. The method of claims 79-91, wherein the emulsion resulting from step (b) is a water-in-oil emulsion.

93. The method of claims 79-91, wherein the emulsion resulting from step (c) is an oil-in-water emulsion.

94. The method of claims 79-93, wherein the polymer of step (b) is a biodegradable polymer.

95. The method of claim 94, wherein the biodegradable polymer is polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid.

96. The method of any one of claims 79-95, wherein the surfactant or stabilizer of step (c) is anionic, cationic, or nonionic.

97. The method of claim 96, wherein the surfactant and/or stabilizer is a poloxamer, a polyamine, PEG, Tween-80, gelatin, dextran, pluronic L-63, pluronic F-68, pluronic 188, pluronic F-127, PVA, PAA, methylcellulose, lecithin, DMAB, PEMA, vitamin E TPGS (D-a-tocopheryl polyethylene glycol 1000 succinate), hyaluronic acid, poly amino acids (e.g polymers of lysine, arginine, aspartic acid, glutamic acid, serine, threonine, tyrosine and cysteine, sodium cholate or their enantiomers), methylcellulose, hydroxyethylcellulose, hydroxyprolylcellulose, hydroxypropylmethylcellulose, gelatin, a carbomer, or a sulfate polymer.

98. The method of any one of claims 79-97, wherein the primary emulsion of step (b) is obtained by homogenization.

99. The method of any one of claims 79-97, wherein the primary emulsion of step (b) is obtained by sonication.

100. The method of any one of claims 79-99, wherein the secondary emulsion of step (c) is obtained by homogenization.

101. The method of any one of claims 79-99, wherein the secondary emulsion of step (c) is obtained by sonication.

102. The method of claim 98 or 100, wherein homogenization is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

103. The method of claim 99 or 101, wherein sonication is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

104. The method of any one of claims 79-103, wherein the hardening of nanoparticles in step (d) is performed by evaporation of the solvent.

105. The method of claim 104, wherein evaporation is active evaporation or passive evaporation.

106. The method of claim 105, wherein active evaporation is vacuum-driven evaporation.

107. The method of claim 106, wherein the vacuum-driven evaporation is performed under high pressure or low pressure.

108. The method of claim 105, wherein passive evaporation is performed by stirring.

109. The method of any one of claims 104-108, wherein evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours.

110. The method of any one of claims 80-109, wherein filtering, washing, and concentrating the nanoparticles in step (e) is performed by filtration, gel filtration, membrane filtration, dialysis, centrifugation, chromatography, density gradient centrifugation, or combinations thereof.

111. The method of any one of claims 79-110, wherein the particles have a negative zeta potential.

112. The method of claim 111, wherein the zeta potential of the particles is between about 0 and −100 mV.

113. The method of claim 112, wherein the zeta potential of the particles is between about −30 and −80 mV.

114. The method of any one of claims 79-113, wherein the particles have a diameter of between about 0.3 μm to 3 μm.

115. The method of claim 114, wherein the particles have a diameter of between about 0.3 μm to 1 μm.

116. The method of claim 115, wherein the particles have a diameter of between about 0.4 μm to 1 μm.

117. The method of any one of claims 79-114, wherein at least 90% of the particles have a diameter of between about 0.3 μm to 3 μm.

118. The method of claim 117, wherein at least 90% of the particles have a diameter of between about 0.3 μm to 1 μm.

119. The method of claim 79-116, wherein at least 90% of the particles have a diameter of between about 0.4 μm to 1 μm.

120. The method of claim 79-114, wherein at least 50% of the particles have a diameter of between about 0.3 μm to 3 μm.

121. The method of claim 120, wherein at least 50% of the particles have a diameter of between about 0.3 μm to 1 μm.

122. The method of claim 121, wherein at least 50% of the particles have a diameter of between about 0.4 μm to 1 μm.

123. The method of any one of claims 79-114, wherein at least 10% of the particles have a diameter of between about 0.3 μm to 3 μm.

124. The method of claim 123, wherein at least 10% of the particles have a diameter of between about 0.3 μm to 1 μm.

125. The method of any one of claims 79-124, wherein one or more MG associated antigen encapsulated within the particle composition is about 0.1 to 100 μg/mg.

126. The method of any one of claims 79-125, wherein one or more MG associated antigens are the AChR protein, muscle tyrosine kinase (MuSK) protein, low-density lipoprotein receptor-related protein 4 (LRP4) protein, agrin protein (AGRN), cortactin protein (CTTN), titin protein (TTN), ryanodine receptor (RYR1, RYR2, RYR3) and/or a portion thereof, or one or more antigenic epitopes thereof.

127. The method of any one of claims 79-126, wherein the antigen is an MG associated antigen selected from the group comprising table 1.

128. The method of any one of claims 79-127, wherein the antigen is the AChR protein subunit and/or a portion thereof, or one or more antigenic epitopes thereof.

129. The method of any one of claims 79-128, wherein the AChR antigenic epitopes are selected from the group consisting of SEQ ID NOs: 1 to 21.

130. The method of any one of claims 79-129, wherein the AChR antigenic epitopes comprise a combination of SEQ ID NO: 3, SEQ ID NO: 5, SEQ ID NO: 8, SEQ ID NO: 13, SEQ ID NO: 19, SEQ ID NO: 20, and/or SEQ ID NO: 21.

131. The method of any one of the preceding claims, wherein the manufacturing batch size is between 0.01 g to 100 kg.

132. A composition comprising particles encapsulating one or more MG associated antigens made by the method of any one of claims 79-131.

133. The composition of claim 132, further comprising a pharmaceutically acceptable carrier, diluent or excipient.

134. The composition of claim 133, wherein the excipients are sucrose, mannitol, and sodium citrate.

135. A pharmaceutical composition comprising negatively charged particles encapsulating Myasthenia Gravis (MG)-associated antigens, sucrose, mannitol, and sodium citrate.

136. A method of treating a subject having Myasthenia Gravis (MG) comprising administering a composition of claim 132 or 135.

137. A composition comprising liposomes encapsulating one or more Myasthenia Gravis (MG) associated antigens, portions, or combinations thereof.

138. The composition of claim 137, wherein the liposome is negatively charged.

139. The composition of claims 137-138, wherein the liposome has a negative zeta potential.

140. The composition of claim 139, wherein the negative zeta potential is between −100 mV to 0 mV.

141. The composition of claim 140, wherein the negative zeta potential is between −80 mV to −30 mV.

142. The composition of claims 137-141, wherein the MG associated antigens are selected from the group comprising table 1.

143. The composition of claim 137-141, wherein the AChR antigenic epitopes are selected from the group consisting of SEQ ID NOs: 1 to 21.

144. The composition of claim 137-141, wherein the AChR antigenic epitopes comprise a combination of SEQ ID NO: 3, SEQ ID NO: 5, SEQ ID NO: 8, SEQ ID NO: 13, SEQ ID NO: 19, SEQ ID NO: 20, SEQ ID NO: 21.

145. The composition of claims 137-144, wherein the size of the liposome is between 100-1000 nm.

146. A method of inducing tolerance in a subject in need thereof comprising administering to the subject a composition of any one of claims 137-145.
